(19) United States
(12) Patent Application Publication
(10) Pub. No.: US 2022/0395578 A1
(43) Pub. Date: Dec. 15, 2022

(54) THERAPEUTIC ANNEXIN-DRUG CONJUGATES AND METHODS OF USE

(71) Applicant: The Board of Regents of the University of Oklahoma, Norman, OK (US)

(72) Inventors: ROGER G. HARRISON, Norman, OK (US); PATRICK H. MCKERNAN, Norman, OK (US); BENJAMIN M. SOUTHARD, Ponca City, OK (US); CHARLES P. BAGARIE, Athens, GA (US)

(21) Appl. No.: 17/621,385

(22) PCT Filed: Jun. 25, 2020
(86) PCT No.: PCT/US2020/039650

§ 371 Date: Dec. 21, 2021

Related U.S. Application Data
(60) Provisional application No. 62/867,971, filed on Jun. 28, 2019.

(51) Int. Cl. A61K 47/64; A61K 45/06; A61P 31/04; A61P 35/00

(52) U.S. Cl. CPC A61K 47/64; A61K 45/06; A61P 31/04; A61P 35/00

(57) ABSTRACT

Therapeutic protein-drug conjugates comprising annexins conjugated to drug payloads for targeting stressed human cells (e.g., cancer cells), bacterial cells, fungal cells, or parasitic cells which express phosphatidylserine. The protein-drug conjugates generally contain multiple drug molecules per annexin molecule. The annexin binds to the surface of cells, but is also endocytosed efficiently, thereby delivering the drug to the cytoplasm of the target cell.

### CROSS-REFERENCE TO RELATED APPLICATIONS

The present patent application claims priority to the provisional patent application filed on Jun. 28, 2019, and identified by U.S. Ser. No. 62/867,971, the entire content of which is hereby incorporated herein by reference.

### BACKGROUND

Antibiotic resistance is a well-known challenge in our modern medical system. A key strategy for combating this challenge is to develop antibiotics that operate via new molecular targets or biological mechanisms.

Eukaryotic and prokaryotic cells share many mechanisms of cell stress and apoptosis. One such conserved element is the expression of phosphatidylserine (PS), an anionic membrane-bound phospholipid, in response to cell stress. This expression of PS is nearly universally conserved, being demonstrated in prokaryotes and eukaryotes. Actively externalized in response to stress, PS is the natural ligand for proteins of the annexin superfamily Interestingly, the expression of annexin superfamily members is also found in prokaryotes and eukaryotes, demonstrating the nature of important mechanisms by which cell stress is recognized, as well as signaled.

In the human body Annexin V is produced by immune cells in order allow them to recognize and bind to stressed cells. In the process of becoming cancerous, tumor cells undergo mutations deleting key cell regulatory elements. The loss of these elements significantly stresses the cell. Annexin binds to stressed cancerous cells. In fact, all cancerous cells of any type bind annexin. The target of annexin V on these stressed cells is the membrane component phosphatidylserine.

Annexin A5 (ANXA5, AV) has been used to deliver therapeutic payloads to PS-expressing cells for managing PS-associated pathologies, including cancer (Neves L F, Krais J J, Van Rite B D et al. Targeting single-walled carbon nanotubes for the treatment of breast cancer using photothermal therapy. Nanotechnology 2013; 24: 375104. Virani N A, Thavathiru E, McKernan P et al. Anti-CD73 and anti-OX40 immunotherapy coupled with a novel biocompatible enzyme prodrug system for the treatment of recurrent, metastatic ovarian cancer. Cancer Lett 2018; 425: 174-82).

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A shows that chemotherapeutic conjugation to ANXA5 increases antibacterial activity of the antibiotic ampicillin (AMP). The annexin 5-AMP conjugate, ANXA5-AMP, is significantly more active than free AMP in culture. We observed a greater than 16,000-fold increase in antibiotic activity as measured by IC50 when AMP is targeted to phosphatidylserine expression with the protein ANXA5 as a delivery vehicle in cultures of L. monocytogenes over 24 h. We furthermore observed a corresponding 3,000-fold decrease in MIC value when AMP (MIC: 1500 mg/L) is targeted to phosphatidylserine expression as part of ANXA5-AMP (MIC: 0.5 mg/L).

FIG. 1B shows the amount of AMP in samples of conjugate as determined by spectroscopic quantification of the yellow fluorescent derivative of AMP, 3,6-diketopiperazine.

FIG. 1C shows the extent of ANXA5 labeling with AMP as confirmed by SDS-PAGE (top). Full bands are isolated to show absence of polymeric byproducts and highlight changes in molecular weight (bottom). R_{f} is calculated as the ratio of the distance migrated by the molecule to that migrated by a marker dye-front. Data are presented as mean±SD (n=3). Statistical significance is noted by ***(p≤0.0005) and **(p≤0.005).

FIG. 2A shows that ANXA5-dependent targeting of PS expression increases antibiotic activity in a positive feedback manner. The enhanced antibiotic activity of the ANXA5-AMP conjugate is not the result of inherent ANXA5-dependent cytotoxicity. No significant decreases in cell growth were detected in cultures of L. monocytogenes treated with the ANXA5 vehicle at concentrations as high as 15 ng/ml. Results depicted as mean±SD (n=3). Statistical significance is noted by ***(p≤0.0005) and **(p≤0.005).

FIG. 2B shows the chemotherapeutic payload of the ANXA5-AMP conjugate upregulates the PS target of delivery. The recruitment of fluorescently labeled ANXA5 to PS in L. monocytogenes is significantly increased in cultures supplemented with ampicillin (10⁻⁵ mg/L). This effect is dependent on Ca²⁺ and is significantly reduced in the presence of the calcium chelator EDTA. Results depicted as mean±SD (n=3). Statistical significance is noted by ***(p 0.0005) and **(p 0.005).

FIG. 2C shows that the PS-dependent increase in conjugate recruitment rapidly induces bacterial death in cultures of L. monocytogenes. In a calcium dependent manner, a significant percentage of bacteria within L. monocytogenes cultures incubated with the ANXA5-AMP conjugate were apoptotic within 3 h as determined by propidium iodide staining. Results depicted as mean±SD (n=3). Statistical significance is noted by ***(p≤0.0005) and **(p≤0.005).

FIG. 2D shows that when the cultures of FIG. 2C were incubated over 24 h, we observed that the activity of the conjugate was significantly decreased in cultures supplemented with 4.5 μM EDTA, highlighting the dependence of this therapeutic on the calcium dependent recognition of PS by ANXA5. Results depicted as mean±SD=3). Statistical significance is noted by ***(p≤0.0005) and **(p≤0.005).

FIG. 3 shows results of a cytotoxicity assay on a 96 plate-wells of EMT6 breast cancer cell line and the conjugate AnnexinA5-Chlorambucil (ANXA5-CHL) and free chlorambucil. The effect of the free chlorambucil is compared to the effect of the conjugate. The two groups were treated at t=0, and the result were read at t=24 hours of the study. Each well was at a confluency of 70% at t=0 with 180 μL of media and 10 μL of chlorambucil or conjugate were added at different concentrations. Viability was determined by the Alamar Blue assay at t=24 hours by fluorescence measurement at 590 nm after an excitation wavelength of 550 nm. The cells treated with free chlorambucil and ANXA5 are respectively represented by ● and ▪ on the graph. They are significantly different with p≤0.005. Data depicted as mean±SE (n=8).

FIG. 4 shows results of a cytotoxicity assay on a 96 plate-wells of 4T1 breast cancer cell line and the conjugate AnnexinA5-Chlorambucil (ANXA5-CHL) and free chlorambucil. The effect of the free chlorambucil is compared to the effect of the conjugate on 4T1 mammary cancer cell line. The two groups were treated at t=0, and the results were read at t=24 hours of the study. Each well was at a confluency of 70% at t=0 with 180 μL of media, and 10 μL of chlorambucil or conjugate were added at different concentrations. Viability was determined by the Alamar Blue assay at t=24 hours by fluorescence measurement at 590 nm after an excitation wavelength of 550 nm. The cells treated with free chlorambucil and ANXA5 are respectively represented by ● and ▪ on the graph. They are significantly different with p≤0.005. Data is presented as mean±SE with n=12.

FIG. 5 shows results of a cytotoxicity assay on a 96 plate-wells of L1210 leukemia cell line and the conjugate AnnexinA5-Chlorambucil (ANXA5-CHL) and free chlorambucil. The effect of the free chlorambucil is compared to the effect of the conjugate on L1210 leukemia cancer cell line. The two groups were treated at t=0, and the results were read at t=24 hours of the study. Each well had at least 25 000 non-adherent cells at t=0 with 180 μL of media and 10 μL of chlorambucil or conjugate were added at different concentrations. Viability was determined by the Alamar Blue assay at t=24 hours by fluorescence measurement at 590 nm after an excitation wavelength of 550 nm. The cells treated with free chlorambucil and ANXA5 are respectively represented by and ▪ on the graph. They are significantly different with p≤0.005. Data depicted as mean±SE (n=8).

FIG. 6 shows results of a cytotoxicity assay on a 96 plate-wells of L1210 Resistant leukemia cell line and the conjugate AnnexinA5-Chlorambucil (ANXA5-CHL) and free chlorambucil. The effect of the free chlorambucil is compared to the effect of the conjugate on L1210 resistant leukemia cancer cell line. The two groups were treated at t=0, and the results were read at t=24 hours of the study. Each well had at least 25 000 non-adherent cells at t=0 with 180 μL of media already containing free chlorambucil at 10 μMol and 10 μL of chlorambucil or conjugate were added at different concentrations. Viability was determined by the Alamar Blue assay at t=24 hours by fluorescence measurement at 590 nm after an excitation wavelength of 550 nm. The cells treated with free chlorambucil and ANXA5 are respectively represented by and ▪ on the graph. They are significantly different with p≤0.005. Data depicted as mean±SE (n=8).

FIG. 7 shows results of a cytotoxicity assay on a 96 plate-wells of P388 Lymphoma cell line and the conjugate AnnexinA5-Chlorambucil (ANXA5-CHL) and free chlorambucil. The effect of the free chlorambucil is compared to the effect of the conjugate on P388 lymphoma cancer cell line. The two groups were treated at t=0 and the results were read at t=24 hours of the study. Each well had at least 25 000 non-adherent cells at t=0 with 180 μL of media already containing free chlorambucil at 10 μMol and 10 μL of chlorambucil or conjugate were added at different concentrations. Viability was determined by the Alamar Blue assay at t=24 hours by fluorescence measurement at 590 nm after an excitation wavelength of 550 nm. The cells treated with free chlorambucil and ANXA5 are respectively represented by ● and ▪ on the graph. They are significantly different with p≤0.005. Data depicted as mean±SE (n=8).

FIG. 8 shows micrographs of 4T1 cells under fluorescence microscopy showing 4T1 cells (A and B) and in-vitro binding specificity ANXA5-CHL on the surface of the cell (C). Observation under a fluorescence microscope (NIKON) 4T1 cell (n=1) without any fluorescence (A). 4T1 cells were originally modified to express a red fluorescent marker, tdTomato 4T1 (B). The Annexin5A is conjugated to a green fluorescent marker (FITC) and bind the cell (C). Fluorescent microscopy showing localization of td Tomato labeled 4T1 cancer cells (B) and FITC-labeled ANXA5-CHL (C). Scale bar is 25 μm.

FIG. 9 shows micrographs of L1210 cell (red) and ANXA5-CHL (green) observed under a fluorescent microscope after photoactivation of the chlorambucil under UV (×100) (A) or only ANXA5-CHL (green) (B). Observation under a fluorescence microscope (NIKON) L1210 cell td Tomato (red). The AnnexinA5 is conjugated to a green fluorescent marker (FITC) and bind the cell (A and B). Scale bar is 25 μm.

FIG. 10 shows an antitumor effect of ANXA5-CHL and chlorambucil against 4T1 tumors on the mammary fat pad number four of Balb/c mice. ANXA5-CHL and free chlorambucil were administered daily at 0.1 mg/mL IP. Treatment occurred for 14 days and it is indicated by the arrow. Data is presented as mean±standard error (n=5). Data is presented as mean±SE (n=12).

FIG. 11 shows results of a long term survival study of Balb/c mice with 4T1 breast cancer cells treated with the conjugate, free chlorambucil or saline solution. Treatment on 4T1/Balb/c mice started 5 days after inoculation with 5×10⁵ 4 T1 mammary breast cancer cells. Mice (n=5) were treated with ANXA5-CHL, free chlorambucil and saline solution daily for 20 days. Survival was monitored and mice were euthanized if there was more than 10% abdominal swelling or mice seems distressed. Data is presented as mean±SE (n=12).

FIG. 12 shows results demonstrating a minimal effect of ANXA5-CHL on 4T1 breast cancer mouse weight. Treatment started 5 days after inoculation with 5×10⁵ 4 T1 mammary breast cancer cells. Tumor weight was measured every 2 days over a 12 days period. Data is presented as mean±SE (n=12).

FIG. 13 shows results of a long term survival study of Balb/c mice with L1210 leukemia treated with the conjugate, free chlorambucil or saline solution. Treatment on L1210/Balb/c mice started 10 days after inoculation with 5×10⁵ L1210 leukemia cancer cells. Mice (n=5) were treated with ANXA5-CHL, free chlorambucil and saline solution daily for 15 days. Survival was monitored, and mice were euthanized if there was more than 10% abdominal swelling or mice seems distressed. Data presented as mean±SE (n=9).

FIG. 14 shows cytotoxicity results for the AV-DM1 (ANXA5-DM1) conjugate and unconjugated DM1 against the EMT6 murine breast cancer cell line. The EC50 is 0.21 nM for the AV-DM1 conjugate and 28 nM for unconjugated DM1. This is an increase in effectivity of 130×.

FIG. 15 shows cytotoxicity results for the AV-DM1 conjugate and unconjugated DM1 against the 4T1 murine breast cancer cell line The EC50 is 0.85 nM for the AV-DM1 conjugate and 320 nM for unconjugated DM1. This is an increase in effectivity of 377×. Data is presented as mean±SE (n=6).

FIG. 16 shows cytotoxicity results for the AV-DM1 conjugate and unconjugated DM1 against the MCF7 human breast cancer cell line. The EC50 is 0.52 nM for the AV-DM1 conjugate and 473 nM for unconjugated DM1. This is an increase in effectivity of 910×. Data is presented as mean±SE (n=6).

FIG. 17 shows cytotoxicity results for the AV-DM1 conjugate and unconjugated DM1 against the P388 murine leukemia cell line. The EC50 is 1.2 nM for the AV-DM1 conjugate and 264 nM for unconjugated DM1. This is an increase in effectivity of 221×. Data is presented as mean±SE (n=6).

FIG. 18 shows cytotoxicity results for the AV-DM1 conjugate and unconjugated DM1 against the L1210 murine leukemia cell line. The EC50 is 0.26 nM for the AV-DM1 conjugate and 93 nM for unconjugated DM1. This is an increase in effectivity of 354×. Data is presented as mean±SE (n=6).

FIG. 19 is a schematic showing the ANXA5-dependent targeting of basal PS expression increases chemotherapeutic cytotoxicity in a positive feedback manner. Targeting PS expression induces a novel apoptotic positive feedback loop in tumor cells. Results depicted as mean±SD (n=4). Statistical significance is noted by ***(p≤0.0001).

FIG. 20 shows the viability of P388 cell cultures treated with the ANXA5-CMB bioconjugate in either Ca²⁺ supplemented medium (4.25 μM) or in EDTA treated Ca²⁺-deficient medium as assayed by calcien-AM and ethidium homodimer staining following a 4 h incubation. Results depicted as mean±SD (n=4). Statistical significance is noted by ***(p≤0.0001).

FIG. 21 shows the cytotoxicity of the protein delivery vehicle ANXA5 in both the L1210 and P388 models of leukemia following a one-day (24 h) incubation as assayed by the resazurin fluorescent metabolic assay. Results depicted as mean±SD=4). Statistical significance is noted by ***(p≤0.0001).

FIG. 22 shows the average cellular expression of PS as assayed by fluorescent ANXA5 labeled with FITC in cultures of P388 following a 6-hour incubation with either control media, or media supplemented with 10 nM CMB. Results depicted as mean±SD (n=4). Statistical significance is noted by ***(p≤0.0001).

### DETAILED DESCRIPTION

Disclosed herein are various embodiments of therapeutic conjugates comprising annexins conjugated to therapeutic drug payloads for targeting stressed human or bacterial cells which express PS. The protein-drug conjugates of the present disclosure in at least certain embodiments comprises multiple drug molecules conjugated to the protein annexin V. Annexin V not only binds to the surface of cells, but is also endocytosed efficiently delivering the drug to the cytoplasm of the target cell.

In one non-limiting embodiment, the therapeutic conjugate is an antibacterial protein-drug conjugate comprised of annexin A5 (ANXA5) linked to an antibiotic such as ampicillin (AMP) (ANXA5-AMP). The ANXA5 serves as a chemotherapeutic delivery vehicle targeting the cell stress-induced expression of the bacterial PS. Localized to the bacterial membrane by ANXA5, the antibiotic AMP thereby induces bacterial cell stress and death. Together these two components create a conjugate compound of unique activity. Evidence indicates that the ANXA5-AMP participates in a novel feedback loop, wherein conjugate recruited by basal bacterial PS expression increases the expression of PS in a cell stress-dependent manner. Induction of PS expression then recruits increasing amounts of the conjugate in a positive feedback loop. In a non-limiting example, a result of this positive feedback loop is that it increases the antimicrobial activity of ampicillin, e.g., against Listeria monocytogenes, by more than 4 orders of magnitude.

In certain embodiments, the Annexin-antibiotic conjugates of the present disclosure have value as treatments against difficult-to-treat intracellular bacterial infections due to facultative or obligate intracellular bacteria that “hide” within cells, including, but not limited to, Mycobacterium tuberculosis, which causes tuberculosis (TB), including drug-resistant TB, Mycobacterium leprae, Salmonella spp., invasive E. coli, Burkholderia pseudomallei, S. aureus, L. monocytogenes, Neisseria spp., Brucella spp., Shigella spp, Chlamydiae, Rickettsia rickettsii. Intracellular parasitic infections which may be treated include, but are not limited to, Toxoplasma spp., Cryptosporidium spp., Plasmodium spp., Leishmania spp., Babesia spp., and Trypanosoma spp., Entamoeba histolytica, and Entamoeba dispar.

In other non-limiting embodiments, the drug component of the protein-drug conjugate is an anticancer drug such as, but not limited to, chlorambucil (designated herein as CHL or CMB). Chlorambucil was the first chemotherapeutic drug ever employed to treat cancer. In modern medicine chlorambucil has remained the standard of care for leukemia for almost one century due to its potent anticancer activity and well documented safety. Annexin and chlorambucil were linked together using 1-Ethyl-3-(3-dimethylaminopropyl) carbodiimide and N-hydroxysuccinimide (EDC/NHS chemistry). We have tested the activity of the resulting annexin-chlorambucil conjugate against breast cancer, leukemia and lymphoma cells lines. The protein-drug conjugate is 100-fold more toxic to tumor cells than free chlorambucil. No increase in toxicity towards healthy cells was observed. Chlorambucil has a carboxylic acid functional group and no primary amine functional group, making it ideal for conjugation to a protein via EDC/NHS chemistry. Chlorambucil conjugated to a protein is still chemically reactive. Additionally, when the conjugate is broken down by the patient's body the primary product is chlorambucil. Chlorambucil is a well-documented chemotherapeutic employed in treating multiple types of cancer. The ANXA5-CMB conjugate of the present disclosure was used as a treatment in mice with syngeneic orthotopic 4T1 breast tumors at a dose of CMB in the conjugate of 0.5 mg/kg mouse weight. At this dose administered daily, the tumor size was significantly reduced, by approximately 5-fold, as compared to similar mice treated with the same dose of free CMB after 9 days. Further results regarding the Annexin A5-chlorambucil conjugate are shown below in Example 2.

In one non-limiting embodiment of the present disclosure, the anticancer drug of the Annexin-drug conjugate is “DM1” or “mertansine.” This drug has been used for the treatment of leukemias and breast cancers. The synthesis and characterization of the drug is quick and scalable. The protein-drug conjugate has shown excellent in vitro results in leukemia and breast cancers. An average of about eight drug molecules were conjugated to a protein. DM1 is an extremely potent microtubule inhibitor that kills cells by mitotic arrest. DM1 is unusable by itself due to high systemic toxicity, but it has shown excellent promise as the active portion of a conjugate that allows targeting and thus localization of its toxic effects to the targeted cells. Further description and results of the Annexin V-DM1 conjugate are shown below in Example 3.

Abbreviations

1-Ethyl-3-[3-dimethylaminopropyl] carbodiimide hydrochloride—EDC; 2-Fluoro-2-deoxyglucose—FDG; Acute myeloid leukemia—AML; Acute lymphocytic leukemia—ALL; AnnexinA5—ANXA5; Adenosine triphosphate—ATP; Chlorambucil—CHL or CMB; Chronic myeloid leukemia—CML; Chronic lymphocytic leukemia—CLL; Dalton—Da; kilo Dalton—KDa; Dimethyl sulfoxide—DMSO; Deoxyribonucleic acid—DNA; Enhanced permeability and retention—EPR; Fluorescein isothiocyanate—FITC; Immunoglobulin—Ig; Isopropyl β-D-1-thiogalactopyranoside—IPTG; Median lethal dose—LD50; Lysogeny broth—LB; Matrix metalloproteinase—MMPs; Molarity—M (unity); N-hydroxysulfosuccinimide-sulfo—NHS; Nickel heads—Ni-NTA resin; N-p-tosyl-L-phenylalanine chloromethyl ketone—TPCK; Phenylmethylsulfonyl fluoride—PMSF; Phosphatidylcholine—PC; Phosphatidylethanolamine—PE; Phosphatidylserine—PS; Polymerase Chain Reaction—PCR; Ribonucleic acid—RNA; Sodium dodecyl sulfate polyacrylamide gel electrophoresis—SDS-PAGE; Tissue Factor—TF; Ultraviolet (1-400 nm)—UV.

Before further description of embodiments of the present disclosure by way of exemplary drawings, experimentation, results, and laboratory procedures, it is to be understood that the embodiments of the present disclosure are not limited in application to the details of compositions and methods set forth in the following description or illustrated in the drawings, experimentation and/or results. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation. Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (4th ed., Cold Spring Harbor Laboratory Press, Cold Spring Harbor, N.Y. (2012) and Coligan et al. Current Protocols in Immunology (Current Protocols, Wiley Interscience (1991-2017)), which are incorporated herein by reference. The nomenclatures utilized in connection with, and the laboratory procedures and techniques of, analytical chemistry, synthetic organic chemistry, molecular and cellular biology, and medicinal and pharmaceutical chemistry described herein are those well-known and commonly used in the art. Standard techniques are used for chemical syntheses, chemical analyses, pharmaceutical preparation, formulation, and delivery, and treatment of patients.

All publications, published patent applications, and issued patents mentioned in the specification are indicative of the level of skill of those skilled in the art to which the presently disclosed inventive concepts pertain. All publications, published patent applications, and issued patents are explicitly incorporated by reference herein to the same extent as if each individual publication, published patent application, or issued patent was specifically and individually indicated to be incorporated by reference.

As utilized in accordance with the present disclosure, the following terms, unless otherwise indicated shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

Throughout this application, the terms “about” or “approximately” are used to indicate that a value includes the inherent variation of error for the composition, the method used to administer the active agent or composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

Where used herein, the terms “specifically binds to,” “specific binding,” “binds specifically to,” and “binding specificity” refer to the ability of a ligand (e.g., an annexin) or other agent to detectably bind to a receptor or a binding epitope while having relatively little detectable reactivity with other proteins, epitopes, or receptor structures presented on cells to which the ligand or other agent may be exposed.

As used herein, the term “nucleic acid segment” and “DNA segment” are used interchangeably and refer to a DNA molecule which has been isolated free of total genomic DNA of a particular species. Therefore, a “purified” DNA or nucleic acid segment as used herein, refers to a DNA segment which contains a coding sequence isolated away from, or purified free from, unrelated genomic DNA, genes and other coding segments. Included within the term “DNA segment,” are DNA segments and smaller fragments of such segments, and also recombinant vectors, including, for example, plasmids, cosmids, phage, viruses, and the like. In this respect, the term “gene” is used for simplicity to refer to a functional protein-, polypeptide-, or peptide-encoding unit. As will be understood by those in the art, this functional term includes genomic sequences, cDNA sequences or combinations thereof. “Isolated substantially away from other coding sequences” means that the gene of interest forms the significant part of the coding region of the DNA segment, and that the DNA segment does not contain other non-relevant large portions of naturally-occurring coding DNA, such as large chromosomal fragments or other functional genes or DNA coding regions. Of course, this refers to the DNA segment as originally isolated, and does not exclude genes or coding regions later added to, or intentionally left in, the segment by the hand of man.

In certain embodiments, DNA sequences in accordance with the present disclosure may include genetic control regions which allow for the expression of the sequence in a selected recombinant host. The genetic control region may be native to the cell from which the gene was isolated, or may be native to the recombinant host cell, or may be an exogenous segment that is compatible with and recognized by the transcriptional machinery of the selected recombinant host cell. Of course, the nature of the control region employed will generally vary depending on the particular use (e.g., cloning host) envisioned.

Truncated genes also fall within the definition of particular DNA sequences as set forth above. Those of ordinary skill in the art would appreciate that simple amino acid removal can be accomplished, and the truncated versions of the sequence simply have to be checked for the desired biological activity in order to determine if such a truncated sequence is still capable of functioning as required. In certain instances, it may be desired to truncate a gene encoding a protein to remove an undesired biological activity, as described herein.

Nucleic acid segments having a desired biological activity may be isolated by the methods described herein. The term “a sequence essentially as set forth in SEQ ID NO:X” means that the sequence substantially corresponds to a portion of SEQ ID NO:X and has relatively few amino acids or codons encoding amino acids which are not identical to, or a biologically functional equivalent of, the amino acids or codons encoding amino acids of SEQ ID NO:X. The term “biologically functional equivalent” is well understood in the art and is further defined in detail herein, as a gene having a sequence essentially as set forth in SEQ ID NO:X, and that is associated with the ability to perform a desired biological activity in vitro or in vivo.

The DNA segments of the present disclosure encompass DNA segments encoding biologically functional equivalent proteins and peptides. Such sequences may arise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the enzyme activity or to reduce antigenicity of the protein or to test mutants in order to examine biological activity at the molecular level or to produce mutants having changed or novel enzymatic activity and/or substrate specificity.

By “protein” or “polypeptide” is meant a molecule comprising a series of amino acids linked through amide linkages along the alpha carbon backbone. Modifications of the peptide side chains may be present, along with glycosylations, hydroxylations, and the like. Additionally, other nonpeptide molecules, including lipids and small molecule agents, may be attached to the polypeptide.

Another embodiment of the present disclosure is a purified nucleic acid segment that encodes a protein that functions in accordance with the present disclosure, further defined as being contained within a recombinant vector. As used herein, the term “recombinant vector” refers to a vector that has been modified to contain a nucleic acid segment that encodes a desired protein or fragment thereof. The recombinant vector may be further defined as an expression vector comprising a promoter operatively linked to said nucleic acid segment.

A further embodiment of the present disclosure is a host cell, made with a recombinant vector comprising one or more genes encoding one or more desired proteins, such as an enzyme conjugate. The recombinant host cell may be a prokaryotic cell. In another embodiment, the recombinant host cell is a eukaryotic cell. As used herein, the term “engineered” or “recombinant” cell is intended to refer to a cell into which one or more recombinant genes have been introduced mechanically or by the hand of man Therefore, engineered cells are distinguishable from naturally occurring cells which do not contain a recombinantly-introduced gene. Engineered cells are thus cells having a gene or genes introduced therein through the hand of man. Recombinantly-introduced genes will either be in the form of a cDNA gene, a copy of a genomic gene, or will include genes positioned adjacent to a promoter associated, or not naturally associated, with the particular introduced gene.

In certain embodiments, the DNA segments further include DNA sequences, known in the art functionally as origins of replication or “replicons,” which allow replication of contiguous sequences by the particular host. Such origins allow the preparation of extrachromosomally localized and replicating chimeric or hybrid segments of plasmids, to which the desired DNA sequences are ligated. In certain instances, the employed origin is one capable of replication in bacterial hosts suitable for biotechnology applications. However, for more versatility of cloned DNA segments, it may be desirable to alternatively or even additionally employ origins recognized by other host systems whose use is contemplated (such as in a shuttle vector).

The nucleic acid segments of the present disclosure, regardless of the length of the coding sequence itself, may be combined with other DNA sequences, such as (but not limited to) promoters, polyadenylation signals, additional restriction enzyme sites, multiple cloning sites, epitope tags, polyhistidine regions, other coding segments, and the like, such that their overall length may vary considerably. It is, therefore, contemplated that a nucleic acid fragment of almost any length may be employed, with the total length preferably being limited by the ease of preparation and use in the intended recombinant DNA protocol.

As used herein, a “protein-drug conjugate” refers to a molecule that contains at least one protein, such as an annexin, and at least one therapeutic moiety such as a drug that is covalently linked to the protein. They may be coupled directly or via a linker and in certain embodiments may be produced by chemical coupling methods or by recombinant expression of chimeric DNA molecules to produce fusion proteins.

As used herein, the terms “covalently coupled,” “linked,” “operably-linked,” “bonded,” “joined,” and the like, with reference to the protein and the drug components of the conjugates of the present disclosure, mean that the specified components are either directly covalently bonded to one another or indirectly covalently bonded to one another through an intervening moiety or components, such as (but not limited to) a bridge, spacer, linker or the like. Operably-linked moieties are associated in such a way so that the function of one moiety is not affected by the other, i.e., the moieties are connected in such an arrangement that they are configured so as to perform their usual function. The two moieties may be linked directly, or may be linked indirectly via a linker sequence of molecule. A non-limiting example of a linkage is the covalent linking of the protein and the drug by a flexible oligopeptide linker.

The term “effective amount” refers to an amount of the conjugate sufficient to exhibit a detectable therapeutic effect when used in the manner of the present disclosure. The therapeutic effect may include, for example but not by way of limitation, reducing the concentration or numbers of a bacterium in a subject's blood, or reducing the number of infected cells in a tissue or erythrocytes in the subject's blood, or extending the survival of the subject, or ameliorating the symptoms of a disease in the subject. The effective amount for a subject will depend upon the type of subject, the subject's size and health, the nature and severity of the condition to be treated, the method of administration, the duration of treatment, the nature of concurrent therapy (if any), the specific formulations employed, and the like. The effective amount for a given situation can be determined by one of ordinary skill in the art using routine experimentation based on the information provided herein.

The term “ameliorate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or symptoms associated therewith, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition or disease, is also a successful treatment outcome. A therapeutic benefit therefore need not be complete ablation or reversal of the malarial infection, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

As used herein, the term “concurrent therapy” is used interchangeably with the terms “combination therapy” and “adjunct therapy,” and will be understood to mean that the patient in need of treatment is treated or given another drug for the disease in conjunction with the conjugates of the present disclosure. This concurrent therapy can be sequential therapy where the patient is treated first with one drug and then the other, or the two drugs are given simultaneously.

The term “pharmaceutically acceptable” refers to compounds and compositions which are suitable for administration to humans and/or animals without undue adverse side effects.

By “biologically active” is meant the ability to modify the physiological system of an organism. A molecule can be biologically active through its own functionalities, or may be biologically active based on its ability to activate or inhibit molecules having their own biological activity.

As used herein, “substantially pure” means an object species is the predominant species present (i.e., on a molar basis it is more abundant than any other individual species in the composition). In certain embodiments, a substantially purified fraction is a composition wherein the object species comprises at least about 50 percent (on a molar basis) of all macromolecular species present. In certain embodiments, a substantially pure composition will comprise more than about 80 percent of all macromolecular species present in the composition, or more than about 85%, or more than about 90%, or more than about 95%, or more than about 99% of all macromolecular species present in the composition.

A “liposome” is a small vesicle composed of various types of lipids, phospholipids and/or surfactant. The components of the liposome are commonly arranged in a bilayer formation, similar to the lipid arrangement of biological membranes.

The term “subject” is used interchangeably herein with the term “patient” and includes human and veterinary subjects. For purposes of treatment, the term “mammal” as used herein refers to any animal classified as a mammal, including (but not limited to) humans, non-human primates, monkeys, domestic animals (such as, but not limited to, dogs and cats), experimental mammals (such as mice, rats, rabbits, guinea pigs, and chinchillas), farm animals (such as, but not limited to, horses, pigs, cattle, goats, sheep, and llamas), and any other animal that has mammary tissue.

The terms “treat,” “treating” and “treatment,” as used herein, will be understood to include both inhibition of cancerous cell growth or bacterial or parasite growth as well as killing parasites and/or infected cells.

The term “receptor” as used herein will be understood to include any peptide, protein, glycoprotein, lipoprotein, polycarbohydrate, or lipid that is expressed or overexpressed on the surface of a cell.

The term “homologous” or “% identity” as used herein means a nucleic acid (or fragment thereof) or a protein (or a fragment thereof) having a degree of homology to the corresponding natural reference nucleic acid or protein that may be in excess of 70%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%. For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross-reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

In one embodiment “% identity” represents the number of amino acids or nucleotides which are identical at corresponding positions in two sequences of a protein having the same activity or encoding similar proteins. For example, two amino acid sequences each having 100 residues will have 95% identity when 95 of the amino acids at corresponding positions are the same.

Another example of a mathematical algorithm used for comparison of sequences is the algorithm of Myers & Miller, CABIOS 1988, 4, 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988, 85, 2444-2448.

Another algorithm is the WU-BLAST (Washington University BLAST) version 2.0 software (WU-BLAST version 2.0 executable programs for several UNIX platforms). This program is based on WU-BLAST version 1.4, which in turn is based on the public domain NCBI-BLAST version 1.4 (Altschul & Gish, 1996, Local alignment statistics, Doolittle ed., Methods in Enzymology 266, 460-480; Altschul et al., Journal of Molecular Biology 1990, 215, 403-410; Gish & States, Nature Genetics, 1993, 3: 266-272; Karlin & Altschul, 1993, Proc. Natl. Acad. Sci. USA 90, 5873-5877; all of which are incorporated by reference herein).

In addition to those otherwise mentioned herein, mention is made also of the programs BLAST, gapped BLAST, BLASTN, BLASTP, and PSI-BLAST, provided by the National Center for Biotechnology Information. These programs are widely used in the art for this purpose and can align homologous regions of two amino acid sequences. In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Specific amino acids may be referred to herein by the following designations: alanine: ala or A; arginine: arg or R; asparagine: asn or N; aspartic acid: asp or D; cysteine: cys or C; glutamic acid: glu or E; glutamine gln or Q; glycine: gly or G; histidine: his or H; isoleucine: ile or I; leucine: leu or L; lysine: lys or K; methionine: met or M; phenylalanine: phe or F; proline: pro or P; serine: ser or S; threonine: thr or T; tryptophan: trp or W; tyrosine: tyr or Y; and valine: val or V.

Where used herein the term “annexin” refers to any of annexins 1-11 and 13, which are more particularly designated as annexins A1, A2, A3, A4, A5, A6, A7, A8, A9, A10, A11, and A13. Annexin I and annexin V where used herein refer to Annexin A1 and Annexin A5, respectively, for example. The annexins contemplated herein further include non-human cognate orthologs of A1-A11 and A13 from non-human vertebrates, including but not limited to, non-human primates, dogs, cats, horses, livestock animals and zoo animals, which may be used for treatment in said non-human mammals in the methods contemplated herein. The annexins contemplated for use herein are discussed in further detail in V. Gerke and S. E. Moss (Physiol. Rev., 82:331-371 (2002)), the entirety of which is expressly incorporated by reference herein.

Anionic phospholipids are largely absent from the surfaces of resting mammalian cells under normal conditions. PS is the most abundant anionic phospholipid of the plasma membrane and is tightly segregated to the internal side of the plasma membrane in most cell types. Recently, it has been discovered that PS is expressed on the outside surface of red blood cells (erythrocytes) that are infected with malarial pathogens (e.g., Plasmodium sp.). The protein-drug conjugates of the present disclosure can therefore be used as an anti-malarial treatment where the drug is cytotoxic.

As noted herein, in one embodiment of the protein-drug conjugate of the present disclosure, the protein is human annexin V. Annexin V (and other annexins) binds with very high affinity to PS-containing phospholipid bilayers. Annexin V may be obtained, for example, as described in U.S. Pat. No. 7,393,833, the entire contents of which are hereby expressly incorporated by reference. Endogenously administered annexin V actively localizes to PS expressing cells in vivo. The annexin portion of the fusion protein selectively binds to PS expressing cells.

In certain non-limiting embodiments, the dosage of the protein-drug conjugate administered to a subject could be in a range of 1 μg per kg of subject body mass to 1000 mg/kg, or in a range of 5 μg per kg to 500 mg/kg, or in a range of 10 μg per kg to 300 mg/kg, or in a range of 25 μg per kg to 250 mg/kg, or in a range of 50 μg per kg to 250 mg/kg, or in a range of 75 μg per kg to 250 mg/kg, or in a range of 100 μg per kg to 250 mg/kg, or in a range of 200 μg per kg to 250 mg/kg, or in a range of 300 μg per kg to 250 mg/kg, or in a range of 400 μg per kg to 250 mg/kg, or in a range of 500 μg per kg to 250 mg/kg, or in a range of 600 μg per kg to 250 mg/kg, or in a range of 700 μg per kg to 250 mg/kg, or in a range of 800 μg per kg to 250 mg/kg, or in a range of 900 μg per kg to 250 mg/kg, or in a range of 1 mg per kg to 200 mg/kg, or in a range of 1 mg per kg to 150 mg/kg, or in a range of 2 mg per kg to 100 mg/kg, or in a range of 5 mg per kg to 100 mg/kg, or in a range of 10 mg compound per kg to 100 mg/kg, or in a range of 25 mg per kg to 75 mg/kg. For example, in certain non-limiting embodiments, the composition could contain protein-drug conjugate in a range of 0.1 mg/kg to 10 mg/kg, or any range comprising a combination of said ratio endpoints, such as, for example, a range of 10 μg/kg to 10 mg/kg.

Examples of bacterial families which contain bacterial species against which the presently disclosed compositions and treatment protocols may be effective include, but are not limited to: Alicyclobacillaceae, Bacillaceae, Listeriaceae, Paenibacillaceae, Pasteuriaceae, Planococcaceae, Sporolactobacillaceae, Staphylococcaceae, Thermoactinomycetaceae, Aerococcaceae, Carnobacteriaceae, Enterococcaceae, Lactobacillaceae, Leuconostocaceae, Streptococcaceae, Caldicoprobacteraceae, Christensenellaceae, Clostridiaceae, Defluviitaleaceae, Eubacteriaceae, Graciibacteraceae, Heliobacteriaceae, Lachnospiraceae, Oscillospiraceae, Peptococcaceae, Peptostreptococcaceae, Ruminococcaceae, Syntrophomonadaceae, Veillonellaceae, Halanaerobiaceae, Halobacteroidaceae, Natranaerobiaceae, Thermoanaerobacteraceae, and Thermodesulfobiaceae.

Specific bacteria that may be treated with the compositions and methods of the present disclosure include, but are not limited to: Enterococcus faecalis, Enterococcus faecium, Staphylococcus aureus, Staphylococcus aureus, Streptococcus pneumonia, Streptococcus mutans, Streptococcus sanguinis, Staphylococcus epidermidis, Bacillus anthracia, Bacillus cereus, Clostridium botulinum, Clostridium botulinum, and Listeria monocytogenes.

While the compositions and methods of the present disclosure have been described in terms of particular embodiments, it will be apparent to those of ordinary skill in the art that variations may be applied to the compositions and/or methods and in the steps or in the sequence of steps of the methods described herein without departing from the concept, spirit and scope of the inventive concepts. All such similar substitutes and modifications apparent to those skilled in the art are deemed to be within the spirit, scope and concept of the inventive concepts as described herein.

In non-limiting embodiments, classes of antibiotic that can be used in an annexin-drug conjugate include aminoglycosides, antimicrobial sulfonamides, beta-lactams, penicillins (e.g., penicillin G), quinolones and their fluoroquinolone derivatives, and many of the polyketides such as the macrolides and tetracycline families of antibiotics. The primary restriction on the use of antibiotics as part of an annexin conjugate is in the chemistry necessary to synthesize the protein-drug conjugate. The synthesis chemistry should not chemically modify the antibiotic in such a way as to unfavorably modify the anti-bacterial activity of the antibiotic.

In certain embodiments, the drug has a single carboxylic or a thiol functional group as a linking moiety. If the drug does not have a carboxylic or thiol functional group, a primary amine can be used as the drug's linking moiety.

In certain embodiments, the fluoroquinolone family of antibiotics, e.g., levofloxacin, can be attached to annexin with linkers such as carbodiimide or hydroxymethyl phosphine and linker esters such as imidoester, NHS-ester, and pentafluorophenyl esters. Another member of the fluoroquinolone family, trovafloxacin, can be linked to annexin. All members of the aminoglycoside family of antibiotics would work with this chemistry. All the quinolone antibiotics would work as part of a conjugate with this chemistry.

Non-limiting List of possible linkers that are suitable for this type of conjugate:

The chemical modification of chemotherapeutic functional groups that directly participate in antibacterial activity can reduce or eliminate the antibacterial activity of the protein-drug conjugate. For instance, the reaction schema should avoid conjugation chemistry that inactivates the antibiotic by damaging pharmacologically activate functional groups. For example, when linking members of the aminoglycoside class to annexin with linkers of the hydrazide class of linkers. This synthesis first requires oxidation of antibiotic sugar glycols using sodium periodate. During this reaction, the ring opening of vicinal diols by sodium periodate damages the aminoglycoside's saccharide rings of the aminoglycoside and reduces the antimicrobial activity of the resulting conjugate. In contrast to the use of hydrazine linkers where the protein can be inactivated by breaking the chemotherapeutics' antibacterial moieties, other linkers can render the conjugate less active by adding moieties that block the antibacterial active site of the antibiotic. For example, the linking process during the conjugation of cephalosporins to annexin with linkers such as the photoreactive diazirine family can sterically block the beta-lactam ring, preventing its reaction with the bacterial enzymes.

Linkers that work with this chemistry may be built of two or more crosslinking moieties that react with different target groups. Many crosslinking agents that fit this description are called click chemistry reagents. Crosslinking moieties that are compatible with this chemistry include carbodiimides, NHS esters, pentafluorophenyl esters, hydroxymethly phosphines, maleimides, haloacetyls, pyridyldisulfides, thiosultonates and vinylsulfones. These crosslinking moieties can be combined together to produce complex linkers that connect functional groups.

Anti-infective drugs which may be used include but are not limited to quinolones (such as nalidixic acid, cinoxacin, ciprofloxacin and norfloxacin and the like), sulfonamides (e.g., sulfanilamide, sulfadiazine, sulfamethaoxazole, sulfisoxazole, sulfacetamide, and the like), aminoglycosides (e.g., streptomycin, gentamicin, tobramycin, amikacin, netilmicin, kanamycin, and the like), tetracyclines (such as chlortetracycline, oxytetracycline, methacycline, doxycycline, minocycline and the like), para-aminobenzoic acid, diaminopyrimidines (such as trimethoprim, often used in conjunction with sulfamethoxazole, pyrazinamide, and the like), penicillins (such as penicillin G, penicillin V, ampicillin, amoxicillin, bacampicillin, carbenicillin, carbenicillin indanyl, ticarcillin, azlocillin, mezlocillin, piperacillin, and the like), penicillinase resistant penicillin (such as methicillin, oxacillin, cloxacillin, dicloxacillin, nafcillinand the like), first generation cephalosporins (such as cefadroxil, cephalexin, cephradine, cephalothin, cephapirin, cefazolin, and the like), second generation cephalosporins (such as cefaclor, cefamandole, cefonicid, cefoxitin, cefotetan, cefuroxime, cefuroxime axetil, cefinetazole, cefprozil, loracarbef, ceforanide, and the like), third generation cephalosporins (such as cefepime, cefoperazone, cefotaxime, ceftizoxime, ceftriaxone, ceftazidime, cefixime, cefpodoxime, ceftibuten, and the like), other beta-lactams (such as imipenem, meropenem, aztreonam, clavulanic acid, sulbactam, tazobactam, and the like), beta-lactamase inhibitors (such as clavulanic acid), chloramphenicol, macrolides (such as erythromycin, azithromycin, clarithromycin, and the like), lincomycin, clindamycin, spectinomycin, polymyxin B, polymixins (such as polymyxin A, B, C, D, E₁ (colistin A), or E₂ (colistin B) and the like) vancomycin, bacitracin, isoniazid, rifampin, ethambutol, ethionamide, aminosalicylic acid, cycloserine, capreomycin, sulfones (such as dapsone, sulfoxone sodium, and the like), clofazimine, thalidomide, or any other antibacterial agent that can be lipid encapsulated. Anti-infectives can include antifungal agents, including polyene antifungals (such as amphotericin B, nystatin, natamycin, and the like), flucytosine, imidazoles (such as miconazole, clotrimazole, econazole, ketoconazole, and the like), triazoles (such as itraconazole, fluconazole, and the like), griseofulvin, terconazole, butoconazole ciclopirax, ciclopirox olamine, haloprogin, tolnaftate, naftifine, terbinafine, or any other antifungal that can be lipid encapsulated or complexed and pharmaceutically acceptable salts thereof and combinations thereof.

According to certain embodiments, the antibiotic drug of the conjugate may include: ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin g, penicillin v, piperacillin tazobactam, ticarcillin clavulanic acid, nafcillin, cephalosporin i generation antibiotics, cefadroxil, cefazolin, cephalexin, cephalothin, cephapirin, cephradine cefaclor, cefamandol, cefonicid, cefotetan, cefoxitin, cefprozil, ceftmetazole, cefuroxime, loracarbef, cefdinir, ceftibuten, cefoperazone, cefixime, cefotaxime, cefpodoxime proxetil, ceftazidime, ceftizoxime, ceftriaxone, azithromycin, clarithromycin, clindamycin, dirithromycin, erythromycin, lincomycin, troleandomycin, cinoxacin, ciprofloxacin, enoxacin, gatifloxacin, grepafloxacin, levofloxacin, lomefloxacin, mozzxifloxacin, nalidixic acid, norfloxacin, ofloxacin, sparfloxacin, trovafloxacin, oxolinic acid, gemifloxacin, perfloxacin, imipenem-cilastatin, meropenem, aztreonam, amikacin, gentamicin, kanamycin, neomycin, netilmicin, streptomycin, tobramycin, paromomycin, teicoplanin, vancomycin, demeclocycline, doxycycline, methacycline, minocycline, oxytetracycline, tetracycline, chlortetracycline, mafenide, silver sulfadiazine, sulfacetamide, sulfadiazine, sulfamethoxazole, sulfasalazine, sulfisoxazole, trimethoprim-sulfamethoxazole, sulfamethizole, rifabutin, rifampin, rifapentine, linezolid, streptogramins, quinopristin dalfopristin, bacitracin, chloramphenicol, fosfomycin, isoniazid, methenamine, metronidazol, mupirocin, nitrofurantoin, nitrofurazone, novobiocin, polymyxin, spectinomycin, trimethoprim, colistin, cycloserine, capreomycin, ethionamide, pyrazinamide, para-aminosalicyclic acid, erythromycin ethylsuccinate, and combinations thereof.

The antibiotic drug may be a “β-lactam antibiotic”, i.e., an antibiotic agent that has a β-lactam ring or derivatized β-lactam ring in its molecular structure. Examples of β-lactam antibiotics include but are not limited to, penams, including but not limited to, penicillin, benzathine penicillin, penicillin G, penicillin V, procaine penicillin, ampicillin, amoxicillin, Augmentin® (amoxicillin+clavulanic acid), methicillin, cloxacillin, dicloxacillin, flucloxacillin, nafcillin, oxacillin, temocillin, mecillinam, carbenicillin, ticarcillin, and azlocillin, mezlocillin, piperacillin, Zosyn® (piperacillin+tazobactam); cephems, including but not limited to, cephalosporin C, cefoxitin, cephalosporin, cephamycin, cephem, cefazolin, cephalexin, cephalothin, cefaclor, cefamandole, cefuroxime, cefotetan, cefoxitin, cefixime, cefotaxime, cefpodoxime, ceftazidime, ceftriaxone, cefepime, cefpirome, and ceftaroline; carbapenems and penems including but not limited to, biapenem, doripenem, ertapenem, earopenem, imipenem, primaxin, meropenem, panipenem, razupenem, tebipenem, and thienamycin; and monobactams including but not limited to, aztreonam, tigemonam, nocardicin A, and tabtoxinine β-lactam.

Examples of cytotoxic drugs that can be used in the protein-drug conjugates of the present disclosure include, but are not limited to, in general, alkylating agents, anti-proliferative agents, tubulin binding agents and the like, for example, the anthracycline family of drugs, the vinca drugs, the mitomycins, the bleomycins, the cytotoxic nucleosides, the pteridine family of drugs, diynenes, and the podophyllotoxins. Examples of those groups include, adriamycin, carminomycin, daunorubicin, aminopterin, methotrexate, methopterin, dichloromethotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine and the like. The drug may be selected from camptothecin, homocamptothecin, colchicine, combretastatin, dolistatin, doxorubicin, methotrexate, podophyllotixin, rhizoxin, rhizoxin D, a taxol, paclitaxol, CC1065, or a maytansinoid, and derivatives and analogs thereof.

The drugs of the conjugates of the present invention may be an antineoplastic agent such as Acivicin; Aclarubicin; Acodazole Hydrochloride; Acronine; Adozelesin; Adriamycin; Aldesleukin; Altretamine; Ambomycin; A. metantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Camptothecin; Caracemide; Carbetimer; Carboplatin; Carmustine; Carubicin Hydrochloride; Carzelesin; Cedefingol; Chlorambucil; Cirolemycin; Cisplatin; Cladribine; Colchicine; Combretestatin A-4; Crisnatol Mesylate; Cyclophosphamide; Cytarabine; Dacarbazine; DACA(N-[2-(Dimethyl-amino)ethyl]acridine-4-carboxamide); Dactinomycin; Daunorubicin Hydrochloride; Daunomycin; Decitabine; Dexormaplatin; Dezaguanine; Dezaguanine Mesylate; Diaziquone; Docetaxel; Dolasatins; Doxorubicin; Doxorubicin Hydrochloride; Droloxifene; Droloxifene Citrate; Dromostanolone Propionate; Duazomycin; Edatrexate; Eflornithine Hydrochloride; Ellipticine; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; Gemcitabine; Gemcitabine Hydrochloride; Gold Au 198; Homocamptothecin; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Mertansine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; PeploycinSulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Rhizoxin; Rhizoxin D; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiocolchicine; Thiamiprine; Thioguanine; Thiotepa; Thymitaq; Tiazofurin; Tirapazamine; Tomudex; TOP53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′ Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N, N′-Bis (2-chloroethyl)-N-nitrosourea (BCNU); N-(2-chloroethyl)-N′ cyclohexyl-N-nitrosourea (CCNU); N-(2-chloroethyl)-N′-(trans-4-methylcyclohexyl-N-nitrosourea (MeCCNU); N-(2-chloroethyl)-N′-(diethyl) ethylphosphonate-N-nitrosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans apthal; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); or 2-chlorodeoxyadenosine (2-Cda).

Other suitable anti-neoplastic compounds include, but are not limited to, 20-pi-1,25 dihydroxyvitamin D3; 5-ethynyluracil; abiraterone; aclarubicin; acylfulvene; adecypenol; adozelesin; aldesleukin; all-tyrosine kinase antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; argininedeaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; basic fibroblast growth factor (bFGF) inhibitor, bicalutamide; bisantrene; bisaziridinylspermine; bisnafide; bistratene A; bizelesin; breflate; bleomycin A2; bleomycin B2; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; and cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones; epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; merbarone; meterelin; methioninase; metoclopramide; MIF inhibitor; ifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues and derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; rapamycin; ras farnesyl protein transferase inhibitors; ras inhibitors; ras-GAP inhibitor, retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. The drug may be an antiproliferative agent, for example piritrexim isethionate, or an antiprostatic hypertrophy agent such as, for example, sitogluside, a benign prostatic hyperplasia therapy agent such as, for example, tamsulosin hydrochloride, or a prostate growth inhibitor such as, for example, pentomone.

In certain embodiments, the presently disclosed drug conjugates may be used in combination with an immunostimulant. The destruction of the tumor cells and/or tumor vasculature causes tumor antigens to be released into the bloodstream. Tumor antigens alone may not be sufficient to stimulate an appropriate immune response. However, the addition of an immunostimulant has been shown to significantly enhance the immune response of the host to the tumor cells, which allows the immune system to mount a systemic attack on the remaining cells of the tumor. Any immunostimulant known in the art or otherwise capable of functioning in accordance with the present disclosure may be utilized in the compositions, methods and kits described herein. Examples of immunostimulants that may be utilized in accordance with the presently disclosed and claimed inventive concept include, but are not limited to, cyclophosphamide, glycated chitosan; muramyldipeptide derivatives; trehalose-dimycolates; and BCG-cell wall skeleton; various cytokines; and combinations and/or derivatives thereof. Dosages of immunostimulants can be in the range of, for example, 0.001 to 100 mg/kg of body weight/day, depending on the method of administration.

The methods described herein may thus include the step of administering an effective amount of an immunostimulant, wherein the immunostimulant is effective in significantly enhancing the immune response of the patient to the tumor cells, and thereby allowing the immune system to mount a systemic attack on the remaining cells of the tumor. The immunostimulant may be administered at the same time as either the drug conjugate, or may be administered before or after the administration of the drug conjugate. Alternatively, the immunostimulant may be administered multiple times to the patient.

In the same manner, the methods described herein may include the step of administering an effective amount of an mTOR inhibitor, wherein the mTOR inhibitor is effective in directly or indirectly decreasing the activity of TOR. The mTOR inhibitor may be administered at the same time as the drug conjugate or may be administered before or after the administration of the drug conjugate. Alternatively, the mTOR inhibitor may be administered multiple times to the patient. Examples of mTOR inhibitors include but are not limited to rapamycin (sirolimus), everolimus (RAD001), temsirolimus (CCI-779), ridaforolimus (deforolimus, AP-23573), metformin, tacrolimus, ABT-578, AP23675, AP-23841, 7-epi-rapamycin, 7-thiomethyl-rapamycin, 7-epi-tromethoxyphenyyl-rapamycin, 7-epi-thiomethyl-rapamycin, 7-demethoxy-rapamycin, 32-demethoxy-rapamycin, 7-desmethyl-rapamycin, 42-O-(2-hydroxy) ethyl-rapamycin, and other analogs of rapamycin (“rapalogs”).

One skilled in the art may make any suitable chemical modifications to the above compounds in order to make reactions of that compound more convenient for purposes of preparing the protein-drug conjugates.

### EXAMPLES

The inventive concepts of the present disclosure will now be discussed in terms of several specific, non-limiting, examples. The examples described below, which include particular embodiments, will serve to illustrate the practice of the present disclosure, it being understood that the particulars shown are by way of example and for purposes of illustrative discussion of particular embodiments of the present disclosure only and are presented in the cause of providing what is believed to be a useful and readily understood description of procedures as well as of the principles and conceptual aspects of the inventive concepts.

### Example 1: Annexin-Ampicillin Conjugate

Methods

Protein Production

Recombinant ANXA5 was produced in BL21(DE3) E. coli (NEB, Ipswich, Ma, USA) transfected with a pET-30 Ek/LIC/ANXA5 plasmid (Novagen, Madison, Wis.) and confirmed to bind PS in a Ca²⁺ manner. Briefly, bacteria were grown in Luria broth medium, and protein production was induced by Isopropyl β-D-thiogalactopyranoside. The resulting protein was purified using an N-terminal His-tag sequence for purification by immobilized metal affinity chromatography (IMAC) with immobilized Ni²⁺ (GE Healthcare Life Sciences, Meadowvale, ON, Canada) and an engineered HRV 3C protease (Thermo Fisher Scientific, Waltham, Mass., USA) cleavage site that cleaves the sequence LEVLFQ↓GP removing the His-tag. The sequence was verified by DNA sequencing at the Oklahoma Medical Research Foundation (Oklahoma City, Okla.). Recombinant protein was confirmed as greater than 95% purity by SDS-PAGE and endotoxin free by limulus assay filter (Thermo Fisher Scientific, Waltham, Mass., USA). Where not specified, materials were obtained from Sigma-Aldrich (St. Louis, Mo., USA).

Conjugate Synthesis

Recombinant ANXA5 is linked to ampicillin via a peptide bond in a high yield series of reactions using a one-pot telescoping synthesis employing click-chemistry crosslinkers. First, the carbolic moiety of the ampicillin's thiazolodine ring is chemically activated with the zero-length linker 1-ethyl-3-(3-dimethylaminopropyl) carbodiimide (EDC) forming an unstable O-acylisourea intermediate stabilized by the displacement of EDC with N-hydroxysulfosuccinimide (sulfo-NHS). In this step, a 6.5 mM solution of ampicillin in 1 mL of 20 mM phosphate buffer at a pH of 7.4 is supplemented with 65 mM of EDC and sulfo-NHS, and then vigorously vortexing for 30 mM at RT. Excess EDC is at this point quenched with 2 mM β-mercaptoethanol for 15 minutes. The solution of amine reactive AMP-NHS is then supplemented with 325 nM ANXA5 and incubated for 1 hour at RT. The resulting ANXA5-AMP conjugate is purified by dialysis using a 25 kDa MWCO regenerated cellulose filter (Thermo Fisher Scientific, Waltham, Mass., USA) against 2 L of 30 mM phosphate buffer for 8 hours. The dialysate is switched twice during this time. The resulting solution is filtered with 0.2 μm PTFE syringe filters (VWR, Radnor, Pa., USA). The sterile solution is then frozen and stored under liquid nitrogen until immediately before use.

Conjugate Characterization

The ANXA5-AMP is characterized by fluorescent assay and SDS-PAGE. The loading of ampicillin onto the ANXA5 is quantified spectroscopically with a fluorescent derivative of ampicillin formed by the formol-catalyzed ring formation of 3,6-disubstituted diketopiperazine. Supplementing 100 μL samples of the conjugate in 20 mM phosphate buffer with 100 μL of 37.5% formaldehyde (VWR, Radnor, Pa., USA) at pH 4.5. and heating at 100° C. for 2 h produces creates this fluorescent derivative (ex/em: 345/420 nm) which is read on an Infinite M1000 microtiter plate reader (TECAN, Männedorf, CHE).

Antibacterial Growth Assay

MIC and IC50 values were determined by broth microdilution in diH₂O as outlined in CLSI methodology unless otherwise specified. The L. monocytogenes strain EGD was originally obtained from P. A. Campbell and stored at 10⁹ CFU·ml⁻¹ and −80° C. in brain heart infusion (BHI) broth. For experiments, bacteria were sub-cultured and incubated at 37° C. with shaking to mid-log phase and then diluted with BHI broth to 5,000 CFU·ml⁻¹ in 96-well plates and treated. All experiments were performed in triplicate, and all sample concentrations were tested four times per replicate. The MIC was reported as the lowest concentration causing no growth as measured by OD_{600 nm} on an Infinite M1000 microplate reader (Tecan, Männedorf, Switzerland). Analysis of IC50 was performed with a variable slope inhibition model using the GraphPad Prism 7 statistical suite.

Binding Affinity and Bactericidal Activity

The recruitment of fluorescently labeled ANXA5 to PS in L. monocytogenes was assayed by flow cytometry. Briefly, samples were incubated with 10⁻⁵ mg/L of AMP and/or 4.5 μM EDTA for 6 h and then pelleted by centrifugation at 5,000 rcf for 5 min at 4° C. At this point, in the case of binding studies, the pellet was suspended in phosphate buffered saline and supplemented with fluorescent ANXA5 (Thermo Fisher Scientific, Waltham, Mass., USA) per supplier instructions. Samples were analyzed on a C6 Accuri flow cytometer (BD Biosciences, San Jose, Calif., USA) and were initially gated on size and then by fluorescence. Alternatively, in the case of bactericidal assay, the pellet was suspended in phosphate buffered saline with calcein-AM/propidium homodimer viability stain (Thermo Fisher Scientific, Waltham, Mass., USA) per supplier instructions. Samples were analyzed by flow cytometry to determine viability.

A non-limiting example of the protein-drug conjugate synthesis method of the present disclosure is shown below:

1) Dissolve 10 mg of an antibiotic in 1 mL of saline buffer
2) Add 1 mg of EDC (The EDC will bind the carboxylic groups of antibiotic increasing their chemical reactivity towards primary amines.)
4) Add 7 mg of suflo-NHS (Sulfo-NHS stabilizes the EDC activated carboxylic groups, increasing the efficiency of the chlorambucil-annexin reaction.)
5) Stir vigorously for 15 minutes
6) Add 2 μL of β-mercaptoethanol (B-mercaptoethanol neutralizes the excess EDC and NHS preventing their interference in downstream reactions.)
8) Add 1 mL of a 1 mg/ml solution of annexin in phosphate buffer (The annexin is kept at a low concentration to prevent precipitation and crosslinking.
9) Stir gently for 12 hours at 4 C
10) Centrifuge for 10 minutes at 7,000 rcf to remove particulates and any precipitated reactants
11) Retain the supernatant and discard the pellet
12) Dialyze the supernatant against 2 L of phosphate buffered saline for 8 hours, switching the dialysate at least twice. (This step removes the rest of the unbound chlorambucil as well as other upstream contaminants such as β-mercaptoethanol.)
13) Filter the dialysate using a 0.2 μm filter 14) Immediately flash freeze under liquid nitrogen and store at −80° C. until immediately before use.

Results

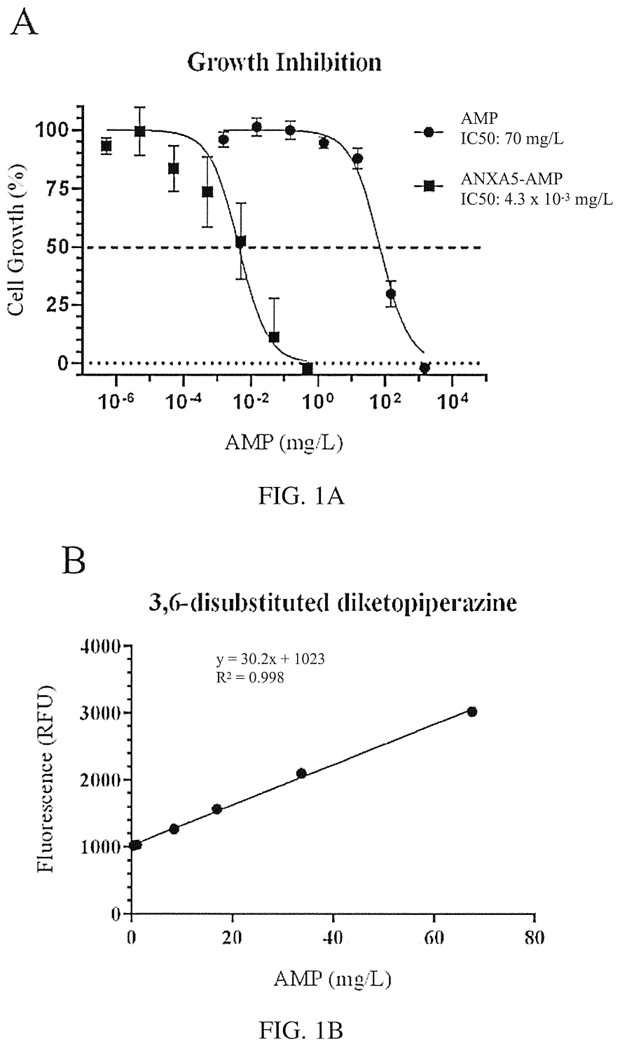

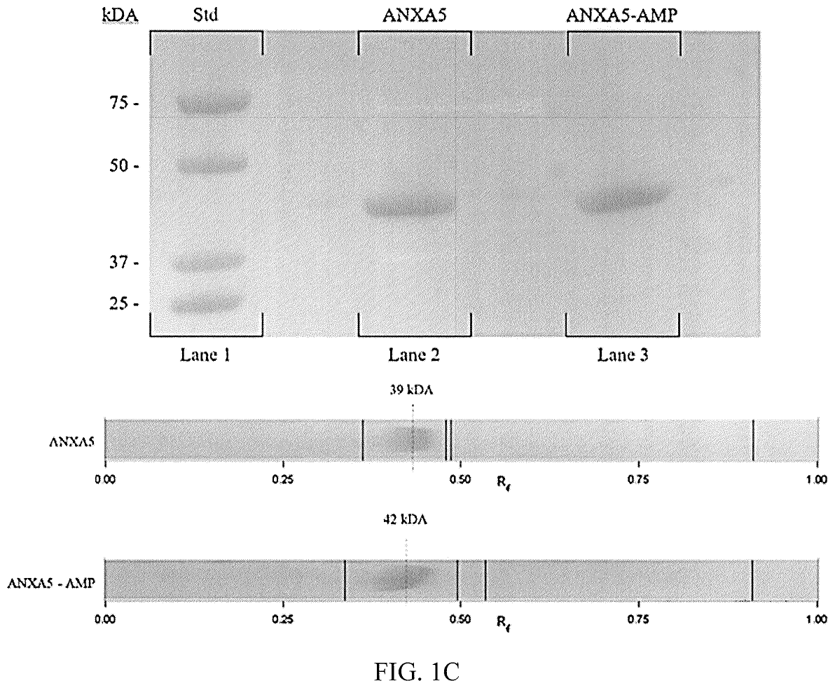

We tested the antibacterial activity of ANXA5-AMP in broth culture against the Gram-positive bacterium L. monocytogenes. We observed that the ANXA5-AMP displayed greater than a 3,000-fold decrease in MIC and a corresponding 16,000-fold decrease in IC50 compared to free ampicillin salt. (FIG. 1A) The concentration of AMP in the ANXA5-AMP conjugate was determined prior to in vitro studies by fluorescent quantification of yellow fluorescent 3,6-disubstituted diketopiperazine, with typical loading rates of 10 mg/L AMP per 1 μg/L of ANXA5 observed (FIG. 1B). Changes in conjugate molecular weight following the addition of AMP to ANXA5 were also confirmed using SDS-PAGE where a roughly 3 kDa shift in molecular weight was observed (FIG. 1C). Using both analytic methods, typical molar loading rates of ˜10 AMP per ANXA5 are observed. No band smearing indicative of polymeric biproducts was observed within SDS-PAGEs, nor was there significant loss of ANXA5 as assayed by Bradford reagent following synthesis.

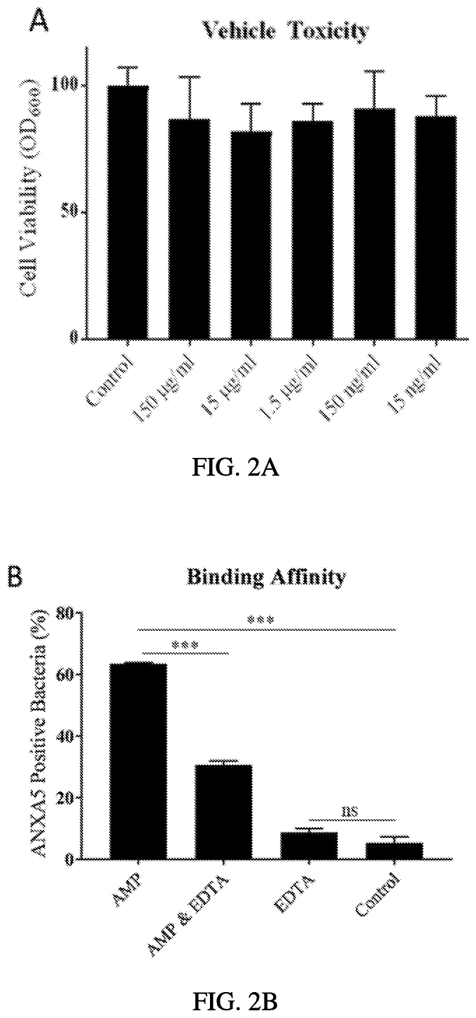

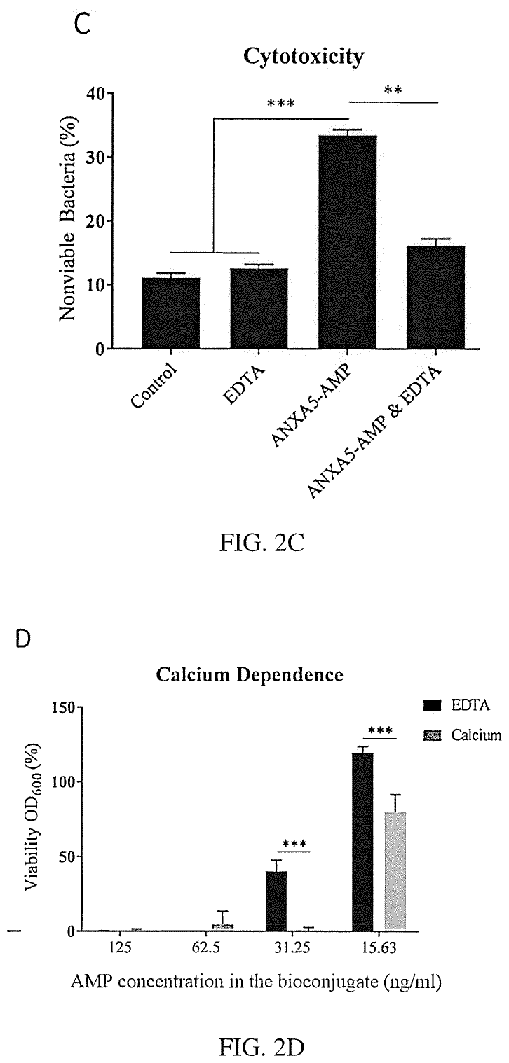

To establish the mechanism of ANXA5-AMP antibacterial activity we undertook several experiments to confirm that ANXA5 recognition of PS was responsible for the antibacterial activity of the conjugate. First, cultures of L. monocytogenes incubated with the ANXA5 delivery vehicle demonstrated no cytotoxicity at any concentration tested. (FIG. 2A). We then confirmed that expression of the molecular target, PS, is upregulated on L. monocytogenes in broth culture when exposed to ampicillin at doses (10⁻⁵ mg/L) much lower than the experimentally determined MIC (FIG. 2B). This is consistent with literature demonstrating that subtoxic concentration of antibiotic induce cellular stress-dependent expression of PS.¹ Furthermore, we confirm that the localization of ANXA5-AMP to PS expression is ANXA5 dependent and the recognition of PS by the protein ANXA5 is calcium-dependent. In cultures of bacteria expressing PS, we do not observe ANXA5 binding in the presence of the Ca²⁺ chelating agent EDTA (4.5 μM). We further demonstrated that that the calcium-dependent accumulation of ANXA5-AMP was necessary to induce death in cultures as assayed by calcein-AM/propidium iodide staining (FIG. 2C). In overnight cultures of L. monocytogenes where the conjugate was supplemented with EDTA (4.5 μM) we observed a decrease in conjugate growth inhibition (FIG. 2D). We conclude that the mechanism of ANXA5-AMP antibacterial activity is dependent on ANXA5 recognition of PS moieties expressed as a result of targeted AMP delivery in a positive feedback cycle.

In summary, the conjugate ANXA5-AMP has significant antibacterial activity. Without wishing to be bound by theory, it is hypothesized that the increase in antibacterial activity results from an induced positive feedback loop in which the conjugate localizes to PS expression, thus delivering ampicillin in a targeted fashion. In this scenario, the localization of AMP to the bacteria induces stress dependent expression of PS, which, in turn, recruits more conjugate. While positive feedback loops such as hypercytokinemia, cell differentiation, and blood clotting are well documented in literature, we believe this is the first time an artificial feedback loop has been employed in such a chemotherapeutic strategy. Given the conservation of PS expression in pathologies such as oncogenesis, and other bacterial/viral/parasitic infections, this demonstrates that a therapeutic modality targeting PS expression on bacterial cells is a viable chemotherapeutic strategy for the treatment of many infectious diseases.

### Example 2: Annexin-Chlorambucil Conjugate

Methods

Production of Recombinant Annexin A5

Recombinant annexin A5 (ANXA5) was produced in BL21(DE3) E. coli transfected with a pET-30 Ek/LIC/ANXA5 plasmid as previously described in Example 1.

Conjugation of Chlorambucil to Annexin Via EDC/NHS Conjugation Method

The most common technique to conjugate a molecule with a carboxylic moiety to an amine-containing molecule is by exposure to a carbodiimide. EDC is a zero-length carboxyl to amine crosslinker with a molecular weight of 191.7 g/mol. This molecule is currently widely used to attach haptens to carrier proteins, crosslink proteins to carboxyl-coated beads or surfaces or form amine bonds in peptide synthesis. The EDC will form an amine reactive O-acylisourea intermediate when it reacts with the carboxylic function of the first molecule. The primary amine bonds the carboxylic acid group by displaced by nucleophilic attack the O-acylisourea, which become an isourea. The carboxylic acid group and amine containing particle are linked by an amide bond and isourea is released from this reaction. However, the O-acylisourea intermediate is unstable in aqueous solutions and an hydrolysis of the intermediate with a regeneration of the carboxyl can occur. The most efficient conditions are an acidic environment with around a 4.5 pH and a buffer without any carboxylic group. However, using a phosphate buffer with neutral pH condition is possible but the efficiency is lower but can be compensated by a higher amount of EDC.

Sulfo-NHS is a compound that can be added to the conjugation reaction to increase efficiency or create stable intermediates. EDC is a coupling reagent and with NHS, they will form an NHS ester, a highly reactive activated and less labile acid intermediate. The intermediate is stable and can be stored at low temperatures. By forming a sulfo-NHS ester, the stability and solubility of the molecule increases but also the efficiency of the conjugation to primary amines at physiologic pH. Lysine is the only amino-acid with a primary amine where the EDC/NHS reaction can occur. They exist also at the end of each polypeptide chain. At physiologic pH, primary amines are positively charged and became more accessible to conjugation reagents. Moreover, they form a nucleophilic group, giving them the ability to be targeted for conjugation. The ANXA5 has an amino-sequence with 22 lysine residues, so theoretically we can conjugate at least 22 molecules of chlorambucil if the steric configuration allows it. However, the secondary, tertiary and quaternary structure of the molecule reduce this possibility. In effect, some of the function would be hidden inside helixes and turns creating the tertiary and quaternary structure of ANXA5.

pET-30 Ek/LIC vector was from EMD Chemicals (Billerica, Mass.). Bovine serum albumin (BSA), Alamar Blue reagent, Triton X-100, EDTA, Dimethyl sulfoxide (DMSO) selenomethionine, isopropyl 2-D thiogalactopyranoside (IPTG), and Tris-acetate-EDTA buffer, N-p-tosyl-L-phenylalanine chloromethyl ketone (TPCK), phenylmethylsulfonyl fluoride were from Sigma-Aldrich (St Louis, Mo.). HRV-3C protease was from Thermo Fisher Scientific (Waltham, Mass.). Sodium phosphate and sodium dodecyl sulfate (SDS) were from Mallinckrodt Chemicals (Phillipsburg, N.J.). The 2 and 100 kDa dialysis membranes were from Spectrum Laboratories (Rancho Dominguez, Calif.). Murine breast cancer cells 4T1 (ATCC® CRL2539™) and EMT6 (ATCC® CRL2755™), Leukemia cells L1210 (ATCC® CCL219™) lymphoma cells P388D1 (ATCC® CCL-46™), RPMI-1640 medium, Waymouth's MB 752/1 Medium, L-glutamine 200 mM, Dulbecco's Modified Eagle's Medium were from ATCC (Manassas, Va.). Fetal bovine serum (FBS) was from Atlanta Biologicals (Lawrenceville, Ga.). Antibiotics, penicillin and streptomycin, were from Invitrogen (Grand Island, N.Y.). His-trap columns were from GE healthcare Chicago, Ill.). Chlorambucil (a.k.a., CMB or CHL) was from TCI America (Portland, Oreg.). HPLC grade ethanol was from Acros Organics (Waltham, Mass.). FITC, Alexa-488, Deep Red Plasma Membrane stain, DAPI, Propidium Iodide, flow cytometry staining buffer, fixation/permeabilization buffer, permeabilization buffer, Slide-A-Lyzer dialysis cassettes (3.5 kDa) were from Thermo Fisher Scientific (Waltham, Mass.). Trypton, yeast extract, and kanamycin monosulfate were obtained from Alfa Aesar (Haverville, Mass.). Sodium hydroxide, potassium chloride, and sodium chloride were from VWR inc. (Radnor, Pa.). HRV-C3 protease was from Sino biologics (Wayne, Pa.). Bradford Reagent were from BioRad (Hercules, Calif.).

Synthesis of Annexin A5—Chlorambucil Conjugate

Chlorambucil readily dissolves under acidic conditions. Initially, a 6.5 mM solution of chlorambucil is prepared in 50 μL of an acid alcohol solution (3% HCl and 95% EtOH v/v, VWR inc., Radnor, Pa., USA). A working solution is then prepared by diluting this acid alcohol solution with 1 mL of 20 mM phosphate buffer (Mallinckrodt Chemicals, Phillipsburg, N.J., USA) at pH 7.4. Supplementing this working solution with 65 mM of both 1-ethyl-3-(3-dimethylaminopropyl) carbodiimide (EDC, Sigma-Aldrich, St. Louis, Mo., USA) and N-hydroxysulfosuccinimide (sulfo-NHS, Sigma-Aldrich, St. Louis, Mo., USA), and then vigorously vertexing for 15 min at room temperature, the butanoic acid moiety of chlorambucil is then chemically activated. At this point excess EDC is neutralized with 2 mM (3-mercaptoethanol (Sigma-Aldrich, St. Louis, Mo., USA). The resulting solution of CMB-NHS is then immediately titrated to a pH of 7.4 with 1 mM NaOH (Mallinckrodt Chemicals, Phillipsburg, N.J., USA) to prevent the acid catalyzed degradation of CMB. This neutral solution of amine reactive CMB-NHS is then supplemented with 2 mL of 975 nM ANXA5 (final concentration 650 nM) and incubated for 12 h at 4° C. At this point, excess chlorambucil forms a visible precipitate in the neutral pH solution and is removed from solution by centrifuging for 1 h at 4° C. and 10,000 rcf. The supernatant is then carefully collected, and the resulting ANXA5-CMB bioconjugate is further purified by dialysis using a 25 kda MWCO regenerated cellulose filter (Thermo Fisher Scientific, Waltham, Mass., USA) against 2 L of 30 mM phosphate buffered saline for 8 h. The dialysate is switched twice during this time. The resulting solution is then sterilized by filtration using a 0.2 μm PTFE syringe filter (VWR Inc., Radnor, Pa., USA). The sterile solution is then flash frozen under liquid nitrogen and stored at −80° C. for up to 1 month or can be used within 12 hours.

Quantification of Chlorambucil Per Annexin A5

A standard curve is established by adding different concentrations of chlorambucil into phosphate buffer with 50% dimethyl sulfoxide (DMSO) to increase the solubility of the chlorambucil. The different concentrations are 0.2 μg/μL, 20 and 200 μg/μL into a 1 mL centrifuge tube. Those tubes are lit by UV light for 30 minutes and then, left 20 min to cool down, and the fluorescence is read at 434 nm. The emission fluorescence is plotted against the concentration of chlorambucil. To determine the concentration of chlorambucil conjugated to the protein, 500 μL of our conjugate is added to a microcentrifuge tube with 50% DMSO. The DMSO is an organic solvent and can precipitate, denature or crystallize proteins but the quantity of chlorambucil stays the same after the DMSO is added to the microcentrifuge tube. After 30 min under UV light and 20 min to cool, the fluorescence is read with the multiplate reader at 434 nm. The results are directly compared to the standard curve to determine the concentration of chlorambucil in our conjugate solution and determine how many molecules of chlorambucil are on each protein of annexinA5.

Cell Lines and Culture Conditions

In-Vitro Experiments: Breast Cancer Cell Line and Culture Conditions

Two breast cancer cell lines were chosen to study the cytotoxicity of the chlorambucil. The first cell line, EMT6 (ATCC® CRL2755™), is a common cell line to study breast cancer taken from mice. They are epithelial cells from breast tissue with a mammary carcinoma and growing in adherent conditions. The second, 4T1 (ATCC® CRL2539™) is a mammary carcinoma breast cancer cell line from BALB/cfC3H from the mammary gland of an animal stage IV human breast cancer. They are also adherent cells. 4T1 mammary carcinoma are highly tumorigenic and invasive, and they can spontaneously metastasize in the early stage of the primary tumor in the mammary gland to multiple distant sites: lymph nodes, blood, liver, lung, brain and bones.

The EMT6 cells were grown in 85% Waymouth's MB 752/1 Medium with 2 mM L-glutamine and 15% fetal bovine serum, at 37° C. with 5% CO₂. The cryopreservation medium is the complete culture medium with 5% DMSO. The murine breast cancer cells 4T1 cells were grown in RPMI-1640 medium enriched with 10% FBS and penicillin/streptomycin antibiotics (100 U/ml and 100 μg/ml, respectively), at 37° C. with 5% CO₂. The cryopreservation medium is the complete culture medium with 5% DMSO.

In-Vitro Experiments: Leukemia Cell Line and Culture Conditions

The L1210 (ATCC® CCL219™) is a lymphocytic leukemia cell line grown in suspension. They are they are skin cells from DBA subline 212. L1210 cells were grown in Dulbecco's Modified Eagle's Medium enriched with 10% horse serum at 37° C. with 5% CO₂. The cryopreservation medium is the complete culture medium with 5% DMSO. Lymphoma cell line P388D1 (ATCC® CCL46™) are monocytes, macrophages growing in suspension. The P388D1 cells were grown in Dulbecco's Modified Eagle's Medium enriched with 10% horse serum at 37° C. with 5% CO₂. The cryopreservation medium is the complete culture medium with 5% DMSO. Those two cell lines are murine models used to evaluate anticancer activity and develop new drugs. Some advantages are that they grow rapidly, homogeneously and are easily reproducible.

In-Vitro Experiments: Cytotoxicity Studies on Cancer Cell Line

Alamar blue assay was used to determine the cytotoxicity of the chlorambucil compared to AnnexinA5-Chlorambucil on each cell line over 20 hours and 4 more hours for the Alamar blue assay in 96 well plate.

In Vitro Fluorescence Visualization

4T1-Td cells (ATCC® CRL2539™) cells were grown until 70% confluence on cover slips. AnnexinA5 (1.5 mg/mL) were tagged with FITC and incubated with the cells for 2 hours, followed with PBS washing of any unbound particle. Cells were fixed in 4% paraformaldehyde, and images were taken on a Nikon Fluorescence microscope. The same protocol was used for the L1210 cells.

Cell Viability Assay

Cells (1000 cells/well) were seeded and cultured for 48 h in 96 well microtiter plates. Cells were then treated for 24 h. Following incubation cell viability was assayed by AlamarBlue assay (Thermo Fisher Scientific, Waltham, Mass., USA) as per manufacturer instructions using a Synergy HTX multi-mode microtiter plate reader (BioTek, Winooski, Vt., USA). In calcien-AM/ethidium homodimer viability assays cells (1000 cells/well) were seeded and cultured for 48 h in 96 well microtiter plates. Cells were then treated for 4 h with protein-drug conjugate, and whole well cellular viability was assayed with calcien-AM/ethidium homodimer (Thermo Fisher Scientific, Waltham, Mass., USA) as per manufacture instructions using a Nikon Eclipse E800 microscope.

In-Vivo Experiments: 4T1 Breast Cancer Cell Line

All procedures complied with a protocol approved by Institutional Animal Care and Use Committee (IACUC) of the University of Oklahoma Health Sciences Center. BALB/c female mice 6 weeks of age, weighing 18-20 g were used. Mice were on a standard chow diet. Mice were injected with 5×10⁴ 4 T1 cells in mammary fat pad number four. Cells were suspended in 50 μL PBS. Mouse body weight was monitored every 3-4 days. Mice bearing tumors were randomized into groups (5 per group) prior to initiation of treatment when tumors reached 100 mm³. ANXA5-CHL fusion protein (200 μL at 1 mg/mL) was administered over 21 days daily and started 5 days after the injection of 4T1 cells. Mice were euthanized once ascite development occurred or animals seemed distressed, and tumor, blood, and organs were collected. Tumor volume was calculated with the modified ellipsoid formula volume=(1/2)×(length×width²) using caliper measurements of the longest dimension and perpendicular width.

In-Vivo Experiments: Leukemia Cell Line

All procedures complied with a protocol approved by Institutional Animal Care and Use Committee (IACUC) of the University of Oklahoma Health Sciences Center. DBA female mice 6 weeks of age, weighing 18-20 g were used. Mice were on a standard chow diet. Mice were injected with 5×10⁵ L1210 cells by intraperitoneal injections. Cells were suspended in 50 μL PBS. Mouse body weight was monitored every 3-4 days. Mice were randomized into groups (5 per group) prior to initiation of the treatment 4 days after the inoculation. ANXA5-CHL fusion protein (200 μL at 1 mg/mL) was administered over 21 days daily and started 48 hours after the injection of L1210 cells. Mice were euthanized once animals seemed distressed, weak and swelling, and tumor, blood, and organs were collected.

Statistical Analysis

Data was analyzed with Excel 2019, Graphpad Prism 8™ software and FIJI. Statistical significance of cytotoxicity results was assessed using a one-way ANOVA and Tukey-Kramer multiple comparisons test. Statistical significance of survival curves was determined by the Gehan-Breslow-Wilcoxon test and Mantel-Haenszel log-rank test. Multiple comparisons were done by using the Bonferroni threshold with a number of samples n=5. Errors are represented graphically as standard error, or SE.

Results

Determination of the Concentration of Chlorambucil

Chlorambucil with a molecular weight of 304.212 g/Mol (Da) was conjugated to the ANXA5 protein. The EDC/NHS protocol conjugates the carboxylic function of the chlorambucil to the amine functions on the ANXA5. By estimating the average weight of the ANXA5-CHL at 39 kDa, around 10 molecules of chlorambucil are fixed by amide bonds on the protein. However, another technique was also used to determine the concentration of chlorambucil presents in the ANXA5 solution after conjugation.

To find the concentration of chlorambucil in the conjugate solution we used a fluorescent microscopy after photoactivation. Following the assay procedure described above, a standard curve is first made with three different concentrations of chlorambucil, respectively 0.2, 2 and 20 mg/mL of chlorambucil. After excitation at 358 nm, the fluorescence is read at 434 nm and the standard curve is made, and we found a linear relationship (y=172.2 ln(x)+725.31) between the fluorescence and the concentration. Then, we read the fluorescence of our conjugate after photoactivation and used the standard curve to determine the concentration of the alkylating agent in our conjugate sample solution. The ANXA5 (36 kDa) has a concentration of 0.1 mg/mL so 2.7 μM and we read a concentration of chlorambucil at 9 mg/mL so 27 μM. Thus 10 molecules of chlorambucil are linked to each molecule of ANXA5.

In-Vitro Results of Cytotoxicity Assays

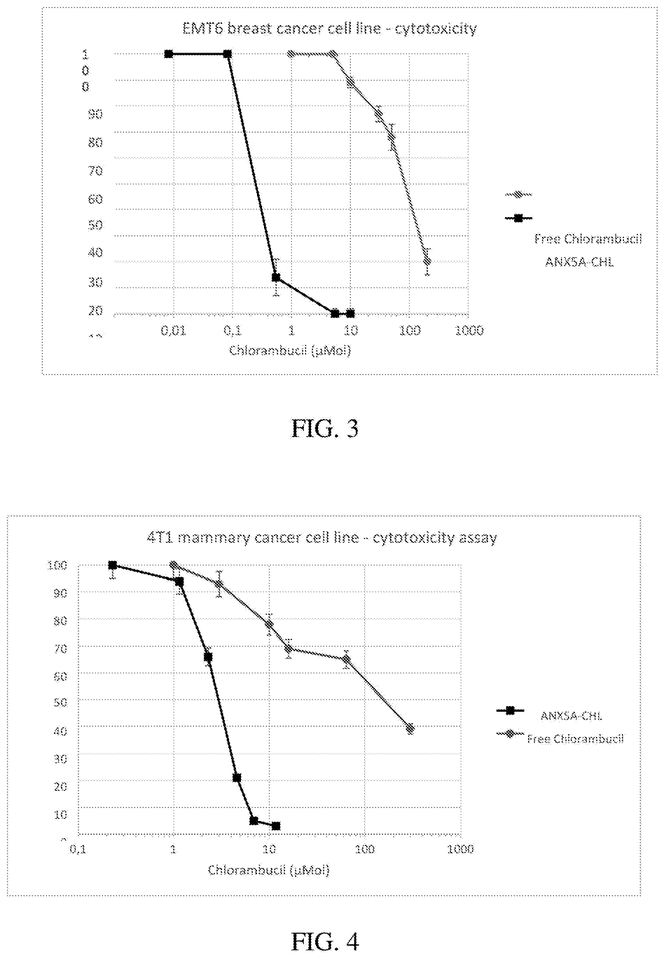

Cytotoxicity studies indicated a significant cytotoxic effect of the ANXA5-CHL molecule on breast cancer cell lines EMT6 and 4T1, leukemia cell line L1210 and Lymphoma cell line P388. The results of the cytotoxicity experiments, with a duration of 16 hours, is shown for the four cell lines in FIGS. 3-7. The conjugate treatment was particularly effective for each cell line. For the leukemia cell line L1210 and lymphoma P388, cytotoxicity studies show that a free chlorambucil concentration of 50 μMol is not enough to kill 30% of the cells.

Results of In-Vitro Cytotoxicity Assay on EMT6 Breast Cancer Cell Line

The cytotoxicity of the conjugate on the EMT6 is 100-fold better than the free drug as we can see in FIG. 3. The conjugate starts to kill cancer cells at 0.1 μM with a very good toxicity, killing 75% of the cells between 0.1 and 0.5 μM. The free chlorambucil is less effective, starting to be active at 10 μM and requiring a concentration of 200 μM to kill 70% of the cells. Only 5 μM is enough to kill all the cells; however, even with 200 μM, more than 20% of the cells remain. The LD50 are respectively, for EMT6 with the conjugate and the free chlorambucil, 0.3 μMol and 100 μMol. The conjugate is more effective to kill EMT6 cancer cells than the free chlorambucil.

Results of In-Vitro Cytotoxicity Assay on 4T1 Mammary Cancer Cell Line

The cytotoxicity of the conjugate on the 4T1 cells (FIG. 4) is 10-fold better than the free drug. The conjugate starts to kill resistant cancer cells at 0.2 μM and erase them with 10 μM. The LD50 is 1.35 μMol for the conjugate and 182 μMol for the chlorambucil. The free chlorambucil starts to kill cells at 1 μM; however, the cytotoxicity is less efficient and can barely kill 60% of the cells with 300 μM. These cells represent a stage IV breast cancer, in which cells are less sensitive to chemotherapeutic drugs. The conjugate cytotoxicity is less effective but is still more effective than the free chlorambucil.

Results of In-Vitro Cytotoxicity Assay on L1210 Leukemia Cell Line

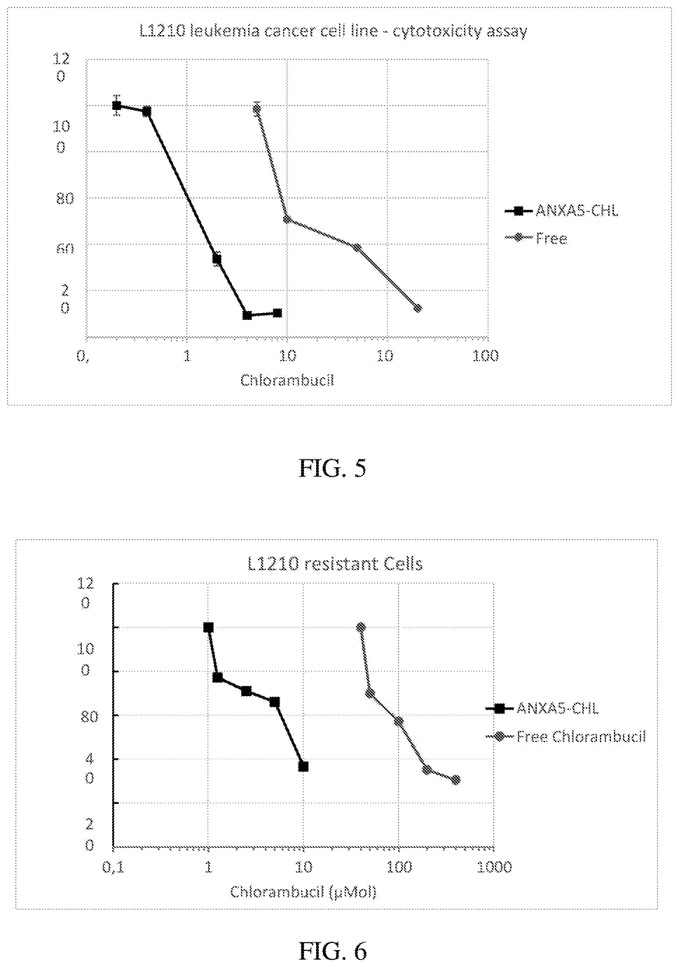

The cytotoxic efficacy of the new conjugate, ANXA5-CHL, was evaluated on L1210 leukemia cells as seen in FIG. 5. The free chlorambucil takes around 60 minutes to bind to the DNA of the L1210 cells. Significant cell death is seen after one day of ANXA5-CHL when only 1 μMol is necessary to start to kill cancer cells, while the free chlorambucil needs a concentration 10 times higher. Only 4 μMol of the conjugate kills 90% of the cancer cells and 200 μMol of free chlorambucil is necessary for the same result, their LD50 are respectively 1.3 μMol and 10 μMol. The conjugate is 10-fold more effective.

Results of In-Vitro Cytotoxicity Assay on L1210 Resistant Leukemia Cell Line

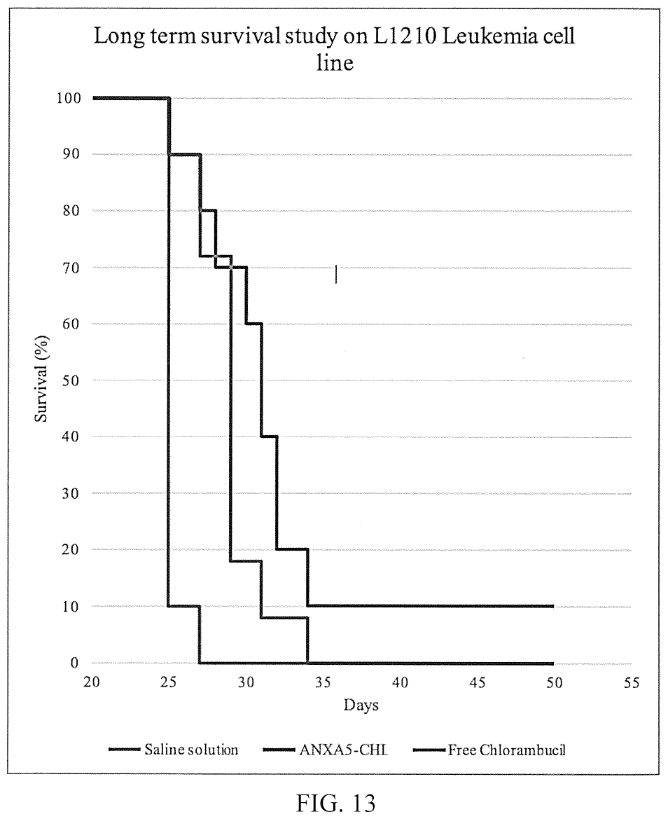

Cells having resistance against chlorambucil should need, in theory, more chlorambucil to be killed. The LD50 for the conjugate is 7.5 μMol and for the chlorambucil alone is 145 μMol. On this FIG. 13 we can see that the difference of the concentration needed to kill cancer cells between the conjugate and the free chlorambucil has decreased: we need 10-times more free chlorambucil as opposite to 100-times more (FIG. 6). However, we were unable to obtain a complete eradication of the resistant leukemia cells, and the concentration of protein in the solution was the maximum, so going higher was impossible. Moreover, the concentration of chlorambucil was the highest, and precipitation was observed with higher concentration.

Results of In-Vitro Cytotoxicity Assay on P388 Lymphoma Cancer Cell Line

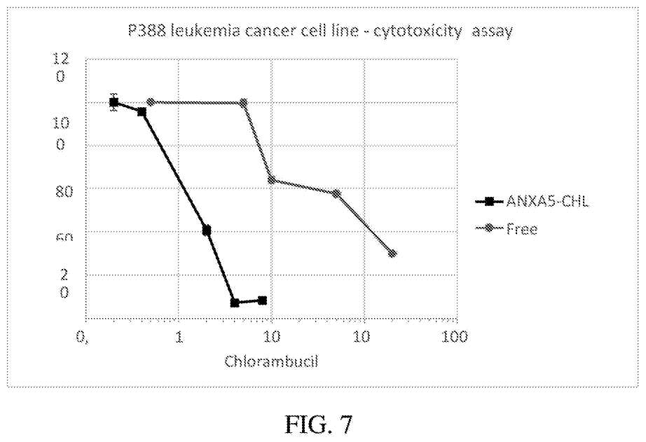

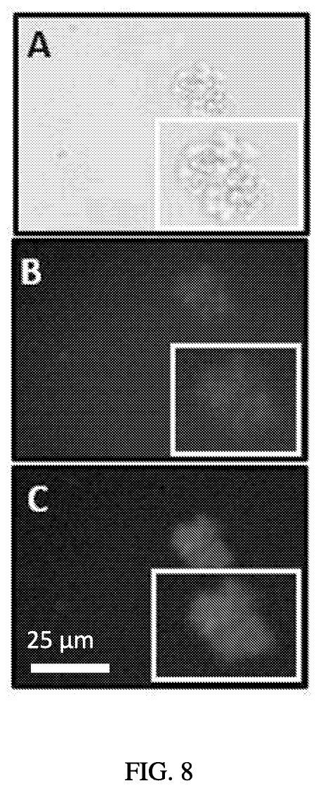

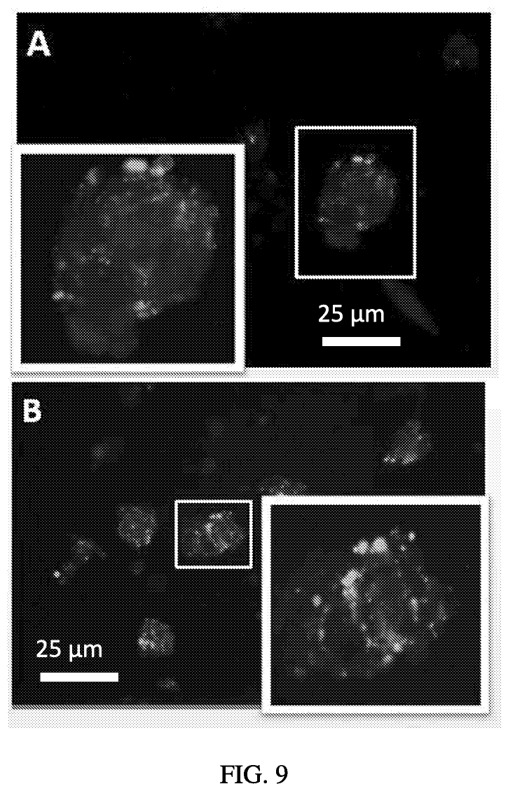

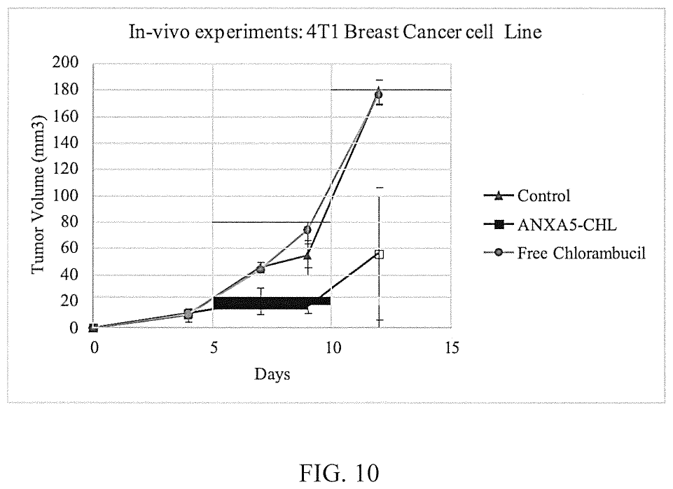

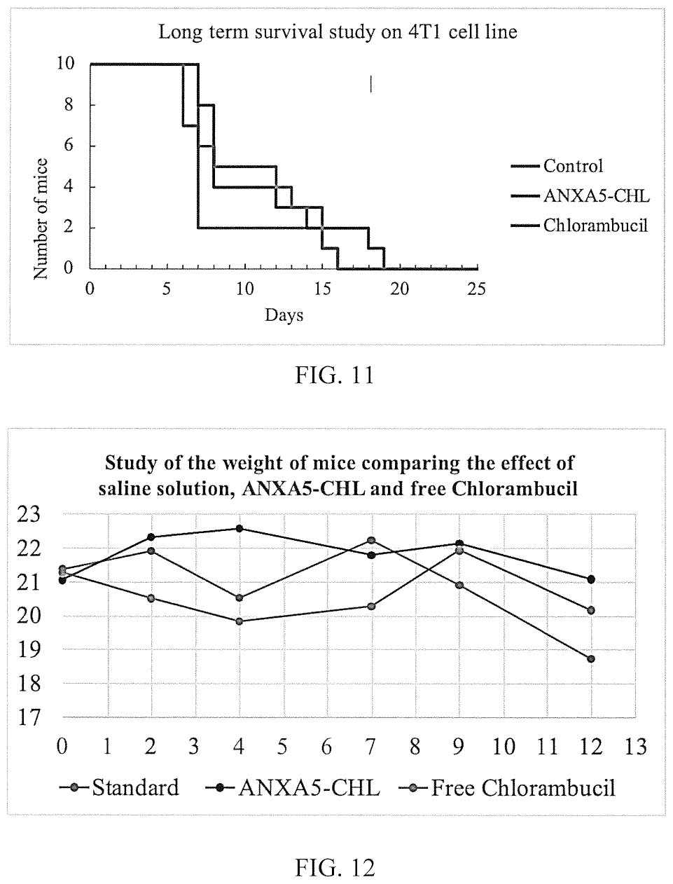

The result of the FIG. 7 of the macrophages of the cell line P388 were used as a complementary cell to study the effect of the conjugate on a different leukemia type. The conjugate is effective at 1 μMol, and its toxicity for cancer cells is higher than the free chlorambucil with more than 90% of the cells dead with 3 μMol. The free chlorambucil is less toxic; it really starts to kill cells at 8 μMol but needs a concentration 14-times higher to be able to kill 75% of the cells. Those results are similar to the leukemia cell line L1210, and this was predictable since these two cell lines evolve in the same way in the body. The LD50 for the P388 cell line is 1.5 μMol for the conjugate and 80 μMol for the free chlorambucil.

Results of In-Vitro Cytotoxicity Assays

The conjugate shows clearly better results than the free chlorambucil on cancer cell lines. The cancer cells can be from a solid tumor, as a breast cancer tumor, or from a non-adherent cancer type, leukemia. This effect could potentially result from active transport across the cell membrane due to the annexinA5. The increased cytotoxicity could be due to a better penetration of the drug induced by the active endocytosis of the ANXA5-CHL into the cells. Chlorambucil alone is clinically limited to leukemia patients too weak to support a strong chemotherapy. The increased cytotoxicity and the targeted system form a better treatment for every kind of patients. The enhancement of the cytotoxic effect on the tumor site or on the cancer cell increases the efficacy of the drug and reduces the therapeutic dose if evaluated in clinical trials.

In Vitro Visualization

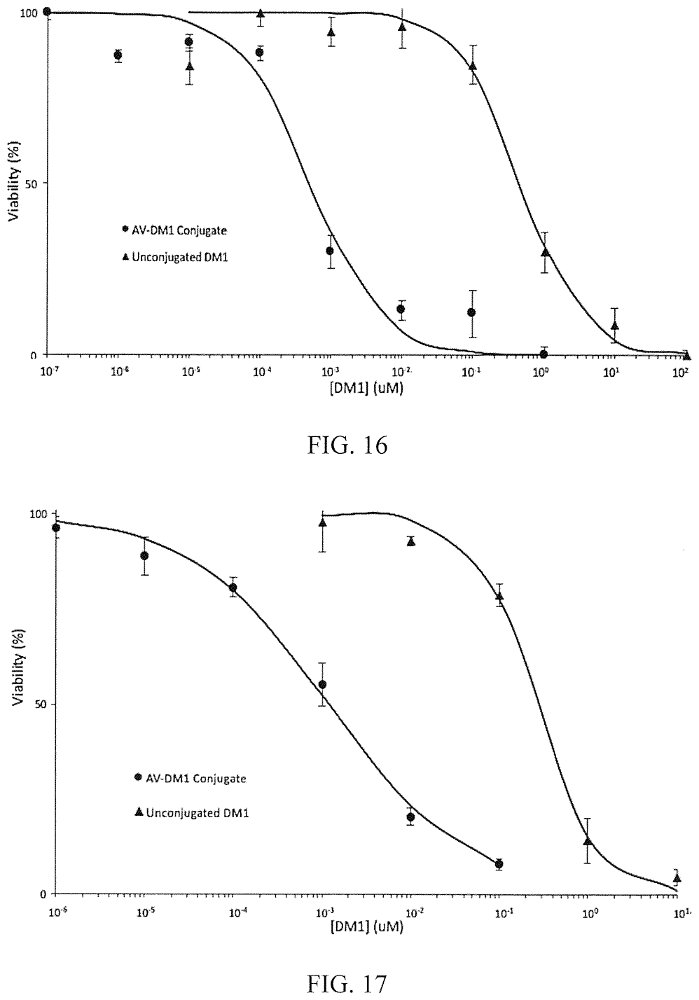

The fluorescence microscope reveals that the conjugate can bind the cancer cells as well as the ANXA5 alone. The mammary cells are around 25 μm and appears red due to the tomato Td modification FIG. 15 (A and B). The 4T1 cells on FIG. 15 show a strong green presence on the cell (red), FITC (Fluorescein isothiocyanate) is really binding on the cell. We can conclude that ANXA5-CHL is binding the cancer cell and is able to bind the solid tumor represented by 4T1 cells. The conjugation technique didn't modify the bound site of the annexinA5 for phosphatidylserine and let the conjugate bond the cell. The leukemia cells L1210 were also red due to the Tomato Td modification (FIG. 16A). After treatment and washing we can visualize more precisely the high presence of ANXA5 on the cell (FIGS. 16A and B). It confirms that the conjugate can bind any cancer cells, adherent or non-adherent from respectively breast cancer and leukemia.

Results of 4T1 In-Vivo Experiments

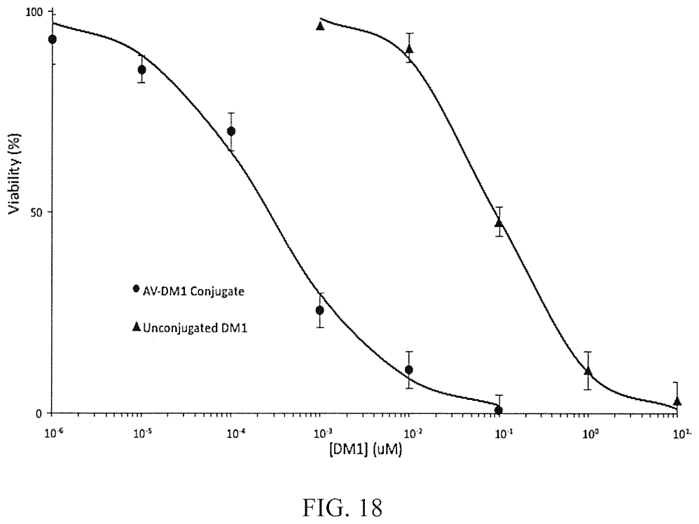

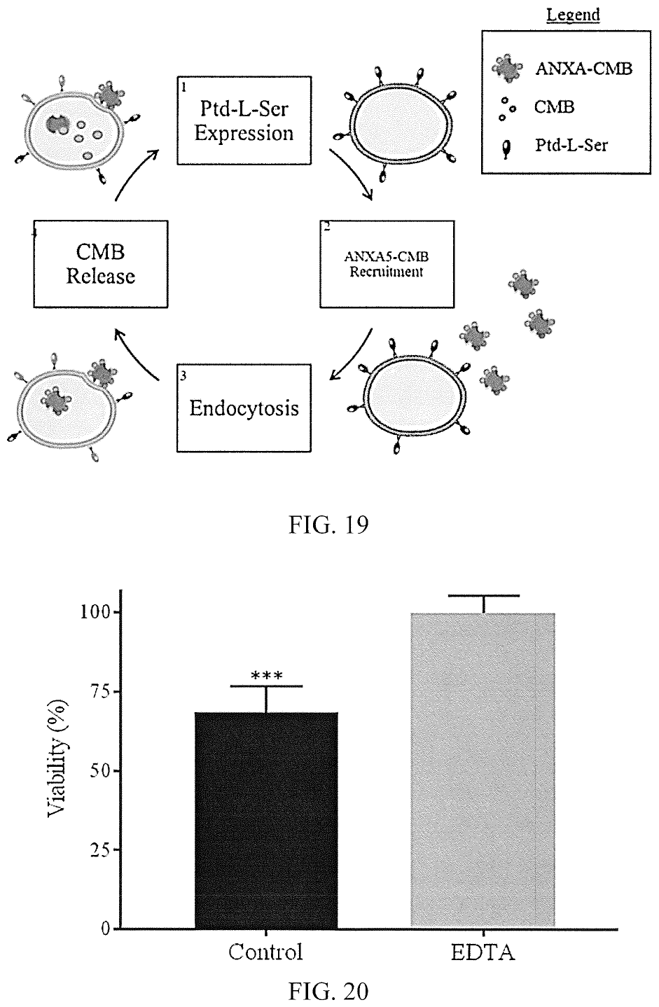

The 4T1 in-vivo experiments last at least 2 months but we stopped the tumor volume and weight measurement after day 12. The conjugate shows promising results as seen on FIG. 17 with a tumor volume clearly reduced compared to the group treated with the saline solution (control) or the free chlorambucil. The ANXA5 reduced the volume increased by 77% compared to the two other groups with a slightly volume increase after day 9 for each group. The volume of the tumor for the control group is 180 mm³ while the volume of the group of mice treated with the conjugate is a third the size (60 mm³). The survival of the mice is also increased with the conjugate, shown on FIG. 18, and indicated a real beneficial effect from the conjugation on the long-term. The survival was monitored to evaluate the efficacy in-vivo of the proposed therapy. As seen in FIG. 18, mice treated with the ANXA5-CHL had a significant increase in survival, 4 more days than free chlorambucil or the control group. The median survival for control group is 8.7 days and 10.4 days for the free chlorambucil. The conjugate has a median survival time of 10.9 days. The ANXA5-CHL increased significantly the survival of the mice with 4T1 breast cancer cell line. As observed on FIG. 19, no negative effects or weight loss was displayed as a result of treatment with the conjugate. The saline solution as expected decreased the average weight of the mice until they lost 10% of their body weight. The conjugate significantly improved the survival of the mice compared to the control group and also inhibited the weight loss induce by the growing tumor because of the small increase of the tumor volume of the solid tumor.

L1210 In-Vivo Experiments

The survival was monitored to evaluate the efficacy in-vivo of the proposed therapy. Almost all the mice with the injection of the saline solution died after day 20, the last one died day 27. The free chlorambucil seems to be more effective and gives 7 more days of survival with a total of 34 days as shown on FIG. 20. The conjugate is more effective on the long-term, but we had to stop the measurement after day 50, indicating that the ANXA5-CHL significantly increased the survival time of the L1210 mice. No side effects have been observed for the ANXA5-CHL.

### Example 3: Annexin-DM1 (Mertansine) Conjugate

Methods

The pET-30 Ek/LIC/ANXA5 plasmid was constructed and sequenced by Oklahoma Medical Research Foundation. The 5 mL HisTrap chromatography column was purchased from GE (Boston, Mass.). HRV 3C protease was purchased from Novagen (Madison, Wis.). Bradford reagent, SDS-PAGE gels, Imperial stain, and Alamar blue dye were purchased from Bio-Rad (Hercules, Calif.). Sulfo-SMCC was purchased from Sigma-Aldrich (St Louis, Mo.). 10 kDa dialysis tubing and DMSO were purchased from Thermo Fisher Scientific (Waltham, Mass.). DM1 (Mertansine) and Live-Dead stain were purchased from Abcam (Cambridge, UK). L1210, P388, EMT6, 4T1 (see Example 2), and MCF7 cell lines were purchased from ATCC (Manassas, Va.). FBS was purchased from Atlanta Biologicals (Lawrenceville, Ga.). Penicillin/streptomycin was purchased from Invitrogen (Grand Island, N.Y.).

Synthesis of Annexin A5-DM1 Conjugate

Production of Annexin A5

Recombinant annexin V (ANXA5) was produced. In brief, E. coli harboring the plasmid containing pET-30 Ek/LIC/ANXA5 were incubated overnight in 100 mL of LB medium with kanamycin. The culture was added to 1 L of fresh LB medium and incubated until the OD of the solution was at 0.5. Protein expression was then induced by adding isopropyl-D-thiogalactopyranoside (IPTG) to the medium and the culture was left to incubate a further 6 hours. The AV-expressing bacteria were centrifuged, collected, and sonicated to lyse the cells. The lysate containing all the cellular proteins including the AV protein with an N-terminal six histidine tail was centrifuged and the debris-free supernatant was collected. The supernatant was put through a nickel HisTrap column and was eluted with a 500 mM imidazole buffer. After dialysis the His-tagged protein was cleaved with the HRV 3C protease and purified again on the HisTrap column and dialyzed against a 20 mM sodium phosphate buffer a final time before being flash frozen in liquid nitrogen. The purified protein was quantified using the Bradford assay and analyzed with SDS-PAGE.

Conjugation of DM1 to Annexin V

First, lysine residues of the ANXA5 protein are modified with a heterobifunctional crosslinking agent. In this step 1.3 μM of ANXA5 and 1.3 mM of amine reactive sulfosuccinimidyl 4-(N-maleimidomethyl) cyclohexane-1-carboxylate (sulfo-SMCC, Thermo Fisher Scientific, Waltham, Mass., USA) are conjugated in deionized water for 1 h at 4° C. on an orbital shaker. The resulting ANXA5-SMCC intermediate is then purified from unreacted SMCC by dialysis overnight against 2 L of 30 mM sodium phosphate buffer at pH 7.4 with a regenerated cellulose dialysis membrane of MWCO 3.5 kDA (Thermo Fisher Scientific, Waltham, Mass., USA). At this point DM1 (Sigma-Aldrich, St. Louis, Mo., USA) is conjugated to the purified sulfur reactive maleimide arm of the ANXA5-SMCC intermediate. In this process 150 μl of 10 mM DM1 in DMSO is added to 2 mL of the purified ANXA5-SMCC solution and the resulting reaction allowed to continue on an orbital shaker for 2 h at 4° C. The resulting ANXA5-DM1 (AV-DM1) conjugate is then purified from excess free DM1 with a second overnight dialysis against 2 L of 30 mM sodium phosphate buffer at pH 7.4 with a regenerated cellulose dialysis membrane of MWCO 3.5 kDA.

Quantification of DM1 in Drug Conjugate

Characterization of Annexin A5-DM1 Conjugate

The conjugate was characterized by SDS-PAGE, and absorbance spectroscopy.

SDS-PAGE

In order to confirm protein modification and estimate the drug loading of the ANXA5 protein, 4-20% 10-well gradient gels were purchased and used with 2× Laemmli sample buffer and tris-glycine-SDS running buffer (TGS). The protein and conjugate were each first denatured by the addition of 2.5% 2-mercaptoethanol and heating at 100° C. for 5 minutes. The samples were run at 200 volts for 25 minutes then stained with Imperial stain and washed in DI water. SDS-PAGE of the conjugate revealed an increase in ANXA5 (Lane 3) molecular weight of approximately 9 kDA following DM1 (MW: 740 Da; 960 Da with crosslinker) covalent addition (Lane 2), corresponding to approximately 9 molecules of DM1 per molecule of ANXA5.

Absorbance Spectroscopy

To determine the average number of DM1 molecules per ANXA5 protein, the absorbance of the a sample of the conjugate and a sample of the same concentration of unconjugated annexin were measured at 288 nm (DM1 peak absorbance). After correcting for the spectral absorbance of ANXA5 as a function of concentration determined by Bradford assay (OD 595 nm), the concentration of DM1 was determined by absorbance (OD: 288 nm). The peaks were subtracted from each other, to find the contribution of only DM1 to the absorbance at 288 nm. The resulting absorbance value was compared to a standard curve of DM1 concentrations in solution to determine the concentration of DM1 on the proteins. The molar concentration of DM1 was divided by the molar concentration of the ANXA5 protein to arrive at the average DM1 per ANXA5 loading.

In Vitro Cytotoxicity

Leukemia

To analyze the in vitro toxicity of the AV-DM1 conjugate compared to unconjugated DM1 in leukemia, two murine leukemia cell lines were used: L1210 and P388. The cells were removed from cryopreservation and cultured in DMEM medium supplemented with 10% FBS and 1% penicillin/streptomycin (Pen/Strep) incubated at 37° C. and 5% CO₂ until one million cells of each strain were ready to be seeded into two 48-well plates, one for each strain. The cells were seeded at a density of 20,000 cells per 500 uL of DMEM medium per well and incubated for 24 hours to allow the cells to return to a proliferative state. The wells were treated in quadruplicate groups with 6 concentrations of both the AV-DM1 conjugate and unconjugated DM1. The AV-DM1 treatment concentrations were from 1 pM to 0.1 uM, and the unconjugated DM1 treatment concentrations were from 1 nM to 10 uM. A control plate with untreated cell controls and no-cell blanks was also prepared. The control and treated plates were incubated for 72-hours at 37° C. and 5% CO₂.

After incubation, 20 uL of alamar blue was added to every plate to a final concentration of 10% in each well. The plates were incubated with alamar blue for 2 hours at 37° C. and 5% CO₂ and analyzed in a plate reader using fluorescence with 530 nm excitation and 590 nm emission. The viability was determined by subtracting the no-cell blank from the untreated cell control and treated experimental plates then dividing the average fluorescence of the treated experimental groups by the average of the untreated cell control.

$$Viability \% = \frac{Treated Fluor - Blank}{Untreated Fluor - Blank} * 100 \%$$

Breast Cancer

To analyze the in vitro toxicity of the AV-DM1 conjugate compared to unconjugated DM1 in breast cancer, three cell lines were used: EMT6 and 4T1 murine breast cancers and MCF7 human breast cancer. Culture medium used for each cell line was different. The medium for EMT6 was Waymouth's medium with 15% FBS, 1% Pen/Strep, for 4T1 was RPMI-1640 with 10% FBS, 1% Pen/Strep; for MCF7 it was EMEM 10% FBS, 1% Pen/Strep. The cells were removed from cryopreservation and cultured in the appropriate medium and incubated at 37° C. and 5% CO₂ until one million cells of each strain were ready to be seeded into two 96-well plates, one for each strain.

The cells were seeded at a density of 12,000 cells per 200 uL of culture medium per well and incubated for 24 hours to allow the cells to return to a proliferative state. The medium was aspirated and replaced with treated media in sextuplicate groups with eight concentrations of both the AV-DM1 conjugate and unconjugated DM1. The AV-DM1 treatment concentrations were from 0.1 pM to 1 uM, and the unconjugated DM1 treatment concentrations were from 10 pM to 100 uM. A control plate with untreated cell controls and no-cell blanks was also prepared. The control and treated plates were incubated for 72 hours at 37° C. and 5% CO₂.

After incubation, the treatment media was aspirated and fresh media with 20 uL of alamar blue was added to every plate to a final concentration of 10% in each well. The plates were incubated with alamar blue for 2 hours at 37° C. and 5% CO₂ and analyzed in a plate reader using fluorescence with 530 nm excitation and 590 nm emission. The viability was determined by subtracting the no-cell blank from the untreated cell control and treated experimental plates then dividing the average fluorescence of the treated experimental groups by the average of the untreated cell control.

Imaging

Live-Dead Stain and Brightfield Images

A fluorescent live-dead stain was used to image P388 cells grown in DMEM media supplemented with 10% FBS and 1% Pen/Strep to about one million total cells. The cell suspension was split, half in one tube and half in another and centrifuged at 1100 RCF for 5 minutes, and the supernatant was removed from each tube. The cell pellet in one tube was resuspended in normal DMEM media; the pellet in the other tube was resuspended in DMEM media with 2 mM EDTA in order to chelate the calcium ions and prevent the calcium dependent binding of AV to PS. The resuspended cells were plated at 200,000 cells per well in a 24-well plate, and treatment with 1 nM AV-DM1 conjugate began immediately. The treated cells were incubated at 37° C. and 5% CO₂ for 3 hours. Brightfield microscope pictures of the cells were taken at 10× magnification and cells were then stained with the Live-Dead stain for 10 minutes and fluorescence microscope pictures were taken at 10× magnification Image composites were made in ImageJ.

Results

AV-DM1 Conjugation

The conjugation protocol described has been performed several times with average yields of about 1 mg of AV-DM1 conjugate with an average drug-protein ratio of about 8. The DM1 standard curve was made by serial dilutions of DM1 in DMSO with a DMSO blank. At high concentrations (<1 mM), the peak absorbance of DM1 is shifted higher to 294 nm and gradually shifts lower to around 288 nm as the concentration becomes less. A standard curve for DM1 in DMSO was determined by taking the absorption values at 288 nm from the above data only until 0.7 mM. Values past 0.7 mM ceased to have a linear relationship between concentration and absorbance. The standard curve equation for Absorbance vs. DM1 concentration was: Abs=2.7907*[DM1]+0.0406. The r² value was 0.98899. The difference in peak absorbances between AV and DM1 is crucial to being able to separate their combined absorbances for analysis of the conjugate. The spectra of both 1 mg/mL AV and 0.15 mM DM1 were taken separately from 200-400 nm. The gap between peaks was approximately 10 nm.

The AV-DM1 conjugate was analyzed to determine the degree of drug loading. The absorbance at 288 nm of AV protein at approximately the same concentration of protein as the AV-DM1 conjugate was subtracted from the absorbance of AV-DM1. The resulting value represented the contribution to the absorption of only the DM1 molecules. The peak absorbance contribution from the DM1 was 0.702 at 288 nm. Using the above standard curve equation, that correlates to approximately 0.24 mM DM1. The conjugate at 0.96 mg/mL is a concentration of 0.027 mM. So in this conjugation, the average drug-protein ratio was 8.9 DM1 molecules per AV protein.

The AV-DM1 conjugate was analyzed by SDS-PAGE on a 4-20% gradient denaturing gel. SDS-PAGE separates proteins based on size. An electric potential is applied to the gel causing proteins to migrate through the gel. Larger proteins, or proteins with additional modifications in this case, are impeded more relative to smaller, or unconjugated, proteins. The AV protein in the left-most lane migrated farther than the AV-DM1 conjugate. Also of note is the absence of any bands at multiples of 36 kDa (72.108 kDa). This is confirmation that no AV-AV polymer products were made in the synthesis.

Size can also be estimated from an SDS-PAGE gel. Protein kDa ladder standard migration distances were compared to the dye migration front and paired to the logarithm of the protein size and plotted. The migration front of the proteins/conjugates with unknown size can be measured and the size can be estimated with the plot made from the protein ladder standards. Once the AV-DM1 conjugate and unconjugated AV sizes were estimated, the difference between them was divided by the combined weight of the linker and drug (about 1 kDa) giving another estimate of the molecules of DM1 per protein. The error associated with SDS-PAGE molecular weight determination is usually within 5-10%. A 5% error associated with the molecular weight of the conjugate is a difference of about 2 kDa, therefore, this method estimated the drug-protein ratio to be 6±2 drug molecules per protein, within the range estimated by the absorption method described.

In Vitro Cytotoxicity

In order to test the toxicity of the AV-DM1 conjugate, several cell lines were treated with many concentrations of the conjugate and compared to treatment with unconjugated DM1. The microtubule inhibiting mechanism of action of DM1 kills cells by mitotic arrest. All five of the cell lines have documented doubling times of 22 or more hours. The standard 24-hour assay would not demonstrate the cytotoxic potential of the drug or conjugate simply because not all cells would have undergone a full cell cycle yet. To demonstrate this, EMT6 cells were used under similar conditions to the 72-hour cytotoxicity assays but treatment was halted after 24 hours and only six of the higher drug concentrations were tested. Neither the drug nor the conjugate showed significant toxicity to EMT6 cells over the 24-hour treatment time. The 72-hour assays proved to be much more effective against all five cell types. The effectiveness of the treatments was assessed by their EC50, the concentration of a drug were 50% effectiveness is reached in a given time period. The lower the EC50, the less of a drug is needed to achieve the half-maximal response. The EC50 is derived from the dose response curves by using the sum of squared differences to fit a sigmoidal regression of the form:

$$V = \frac{Max}{1 + {( \frac{C}{EC 50} )}^{H}} . \qquad Equation 2$$

where V is the response (viability in this context), Max is the theoretical maximum response (100% viability), C is the concentration of drug, EC50 is the concentration of half-maximal effectiveness, and H is the Hill coefficient which describes how “steep” the curve is.

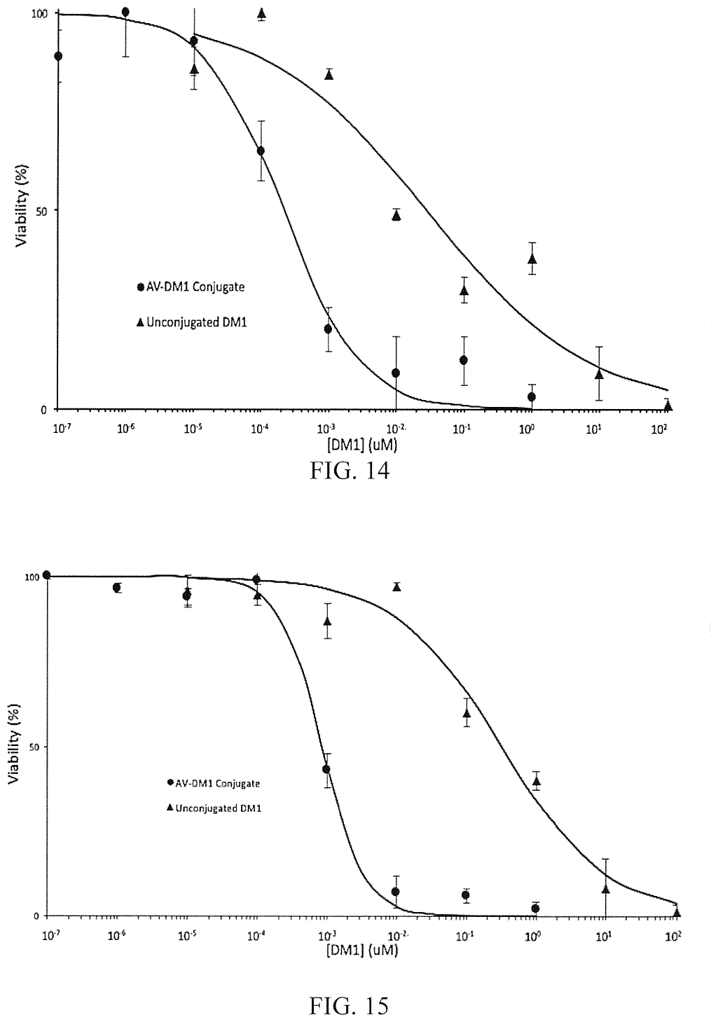

FIGS. 14-18 demonstrate cytotoxicity results for the AV-DM1 conjugate and unconjugated DM1 against various cell lines for leukemia and breast cancer.

FIG. 14 presents cytotoxicity results for the AV-DM1 conjugate and unconjugated DM1 against the EMT6 murine breast cancer cell line. The EC50 is 0.21 nM for the AV-DM1 conjugate and 28 nM for unconjugated DM1. This is an increase in effectivity of 130×.

FIG. 15 presents cytotoxicity results for the AV-DM1 conjugate and unconjugated DM1 against the 4T1 murine breast cancer cell line The EC50 is 0.85 nM for the AV-DM1 conjugate and 320 nM for unconjugated DM1. This is an increase in effectivity of 377×. Data is presented as mean±SE (n=6).

FIG. 16 presents cytotoxicity results for the AV-DM1 conjugate and unconjugated DM1 against the MCF7 human breast cancer cell line. The EC50 is 0.52 nM for the AV-DM1 conjugate and 473 nM for unconjugated DM1. This is an increase in effectivity of 910×. Data is presented as mean±SE (n=6).

FIG. 17 presents cytotoxicity results for the AV-DM1 conjugate and unconjugated DM1 against the P388 murine leukemia cell line. The EC50 is 1.2 nM for the AV-DM1 conjugate and 264 nM for unconjugated DM1. This is an increase in effectivity of 221×. Data is presented as mean±SE (n=6).

FIG. 18 presents cytotoxicity results for the AV-DM1 conjugate and unconjugated DM1 against the L1210 murine leukemia cell line. The EC50 is 0.26 nM for the AV-DM1 conjugate and 93 nM for unconjugated DM1. This is an increase in effectivity of 354×. Data is presented as mean±SE (n=6).

Imaging

Brightfield and fluorescence imaging was done on P388 cells in order to qualitatively analyze both the viability and morphology of cells treated with the AV-DM1 conjugate and to demonstrate the binding specificity of the conjugate. A live-dead stain was used to indicate the viability of the cells. A membrane-permeable non-fluorescent dye that is converted to a green fluorescent form in metabolically active cells stains for viable cells. Propidium iodide is membrane-impermeable red fluorescent dye that binds DNA in cells with damaged membranes. The cells were treated for a period of only 3 hours. This was done in order to maintain the viability of cells in the EDTA-supplemented control group. EDTA chelates calcium ions which prevents efficient binding of the AV-DM1 conjugate, but calcium is also necessary for long term cell viability. Only a small portion of cells in the treatment group would enter metaphase of the mitotic cycle in the 3 hour period, and even fewer would remain arrested there long enough to exhibit signs of apoptosis like membrane damage.

### DISCUSSION

As noted above, the targeted delivery of ANXA5-associated chemotherapeutics to PS expression creates a positive feedback loop where the delivery of drug increases cell stress, thereby increasing PS expression. This process initiates a positive feedback loop, leading to an increase in the cellular expression of PS and a corresponding increase in the recruitment of ANXA5 associated chemotherapeutic. We demonstrate that this novel positive feedback loop based chemotherapeutic strategy induces cell death in hematological malignancies with the drugs chlorambucil and mertansine (DM1).

The cytotoxic activity of both conjugates was compared to that of the free drugs for the P388 and L1210 murine leukemia cell lines. Measuring the IC₅₀ (drug concentration at which the cell viability is inhibited by 50%) in a 24-hour in vitro viability assay, the ANXA5-CMB conjugate was 44- and 17-fold more potent than free CMB in the P388 and the L1210 models, respectively. We further observed that ANXA5-dependent enhancement of chemotherapeutic antineoplastic activity is independent of chemotherapeutic drug class. The antineoplastic activity of the maytansinoid rhizoxin binding site tubulin inhibitor DM1, was significantly enhanced as part of an ANXA5 conjugate as well. In a 72-hour in vitro viability assay, this ANXA5-DM1 conjugate was 208- and 352-fold more potent than free DM1 in the P388 and L1210 models, respectively. Using two drugs of different antineoplastic mechanism of action, the alkylating agent CMB and the microtubule inhibitor DM1, we observed that conjugation of both chemotherapeutics to the protein ANXA5 significantly increases the chemotherapeutics' antineoplastic activity versus free drug.

Having confirmed the broad utility of an ANXA5 based therapeutic strategy, we then proceeded to study the mechanism of cytocidal activity of the conjugates. We hypothesized that an apoptotic positive feedback loop was the mechanism of such increased cytotoxicity (FIG. 19), wherein the ANXA5-CMB conjugate is first recruited to the cell membrane by basal PS expression. The capture of ANXA5-CMB bioconjugate by membrane bound PS then triggers a unique form of endocytosis. During endocytosis within cellular late endosomes, the protein vehicle of the bioconjugate is degraded by lysosomal activity. Simultaneously, the low pH of these vesicles catalyzes the formation of a three-membered aziridinium ring derivative of CMB, increasing its alkylating activity. This activated aziridinium product of CMB cyclization then triggers caspase activation of Xr-family scramblases and ABC-transporters, increasing the expression of PS, thus recruiting ever more conjugate, leading to cell death.

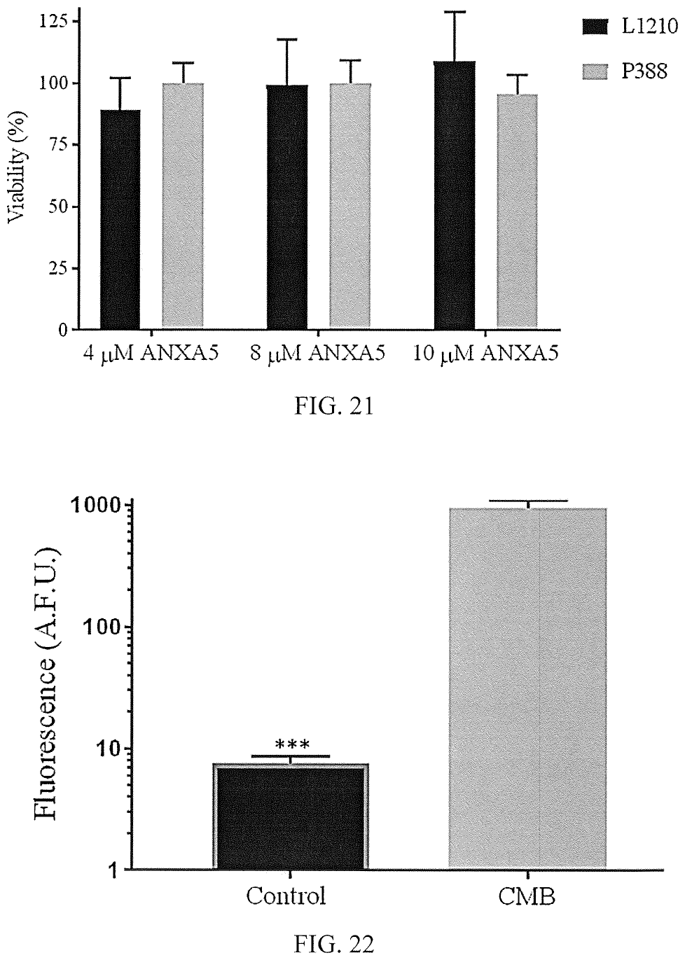

We confirmed this putative feedback mechanism by first establishing the dependence of this unique therapeutic strategy on ANXA5 recognition of PS. The binding of ANXA5 to negatively charged PS is calcium dependent. In the presence of the calcium chelating agent ethylenediaminetetraacetic acid (EDTA), ANXA5-CMB did not significantly affect cellular viability; however, in the presence of control media, supplemented with calcium (4.25 μM) we find that ANXA5-CMB rapidly induces cellular death (FIG. 20). We then confirmed that the ANXA5 delivery vehicle itself poses no significant cytotoxicity in either of the P388 or L1210 cell lines at doses as high as 10 μM (FIG. 21). Finally, we confirmed that CMB even at subtoxic doses is capable of inducing the expression of PS. Cellular PS expression as measured by the ANXA5 affinity is increased by more than 124-fold in the presence of nontoxic doses (10 nM) of CMB (FIG. 22). In, short we demonstrated a unique positive feedback loop by confirming that ANXA5 binding is involved in the mechanism of action, that the ANXA5 vehicle does not induce cell death, and that the chemotherapeutic payload is responsible for the upregulation of PS expression and corresponding increase in ANXA5 recruitment.

One significant advantage of targeting PS expression in leukemia is that the novel positive feedback loop described here specifically targets tumor cells. Rapidly dividing tumor cells are uniquely sensitive to alkylating agents such as the chemotherapeutic payload of the ANXA5-CMB conjugate. CMB recruited to basal PS expression efficiently upregulates PS expression in these tumor cells. In contrast, healthy tissue not undergoing cell division is both naturally more resistant to stress-inducing alkylating agents induced stress, and at the same time not susceptible to ANXA5 binding given that PS is confined to the inner-leaflet of the plasma membrane. We hypothesize that these two factors will together provide a good measure of protection to healthy tissues.

A therapeutic treatment modality employing ANXA5 has several key benefits when compared to other protein-drug conjugates. In contrast to clinically available antibody-drug conjugates (ADCs), such as gemtuzumab ozogamicin, ado-trastuzumab emtansine, brentuximab vedotin, or inotuzumab ozogamicin, we observe that ANXA5 makes for an efficient drug delivery vehicle. While ADCs typically have a low protein:drug molar loading ratios of ˜4, we observe that ANXA5 efficiently loads chemotherapeutics in a protein:drug molar loading ratio up to ˜12. Furthermore, in contrast to current ADCs which target moieties expressed in limited lineages of leukemia and lymphomas, the expression of PS in response to cell stress is not confined to a small subset of neoplasia. With few exceptions, the mechanisms of PS expression in response to chemotherapeutic challenge are universally conserved in hematopoietic and lymphocytic cells tumors. In fact, the stress-induced expression of PS which is critical to this therapeutic strategy is not confined merely to neoplasia of hematological origin.

1. A protein-drug conjugate, comprising: an annexin protein to which is covalently linked at least one therapeutic drug having anticancer, antibacterial, antifungal, and/or antiparasite activity.

2. The protein-drug conjugate of claim 1, wherein the annexin protein is human annexin A5.

3. The protein-drug conjugate of claim 1, wherein the at least one therapeutic drug is an anticancer drug.

4. The protein-drug conjugate of claim 1, wherein the anticancer drug is selected from chlorambucil and mertansine.

5. The protein-drug conjugate of claim 1, wherein the at least one therapeutic drug is an antibacterial drug.

6. The protein-drug conjugate of claim 5, wherein the antibacterial drug is selected from the group consisting of β-lactams, quinolones, sulfonamides, aminoglycosides, tetracyclines, para-aminobenzoic acid, diaminopyrimidines, penicillins, penicillinase resistant penicillins, first generation cephalosporins, second generation cephalosporins, third generation cephalosporins, beta-lactamase inhibitors, chloramphenicol, macrolides, lincomycin, clindamycin, spectinomycin, polymyxin B, polymixins, vancomycin, bacitracin, isoniazid, rifampin, ethambutol, ethionamide, aminosalicylic acid, cycloserine, capreomycin, sulfones, clofazimine, thalidomide, and pharmaceutically acceptable salts thereof and combinations thereof.

7. The protein-drug conjugate of claim 6, wherein the β-lactam is selected from the group consisting of penams, cephems, penems, carbapenems, and monobactams.

8. The protein-drug conjugate of claim 6, wherein the β-lactam is a penam β-lactam selected from the group consisting of ampicillin, penicillin, benzathine penicillin, penicillin G, penicillin V, procaine penicillin, amoxicillin, methicillin, cloxacillin, dicloxacillin, flucloxacillin, nafcillin, oxacillin, temocillin, mecillinam, carbenicillin, ticarcillin, and azlocillin, mezlocillin, and piperacillin.

9. The protein-drug conjugate of claim 1, wherein the at least one therapeutic drug is an antiparasite drug.

10. A therapeutic composition, comprising (1) a protein-drug conjugate comprising an annexin protein to which is covalently linked at least one therapeutic drug having anticancer, antibacterial, antifungal, and/or antiparasite activity, and (2) at least one of an immunostimulant and an mTOR inhibitor.

11. The therapeutic composition of claim 10, wherein the mTOR inhibitor is selected from the group consisting of rapamycin, everolimus, temsirolimus, ridaforolimus, metformin, tacrolimus, ABT-578, AP23675, AP-23841, 7-epi-rapamycin, 7-thiomethyl-rapamycin, 7-epi-tromethoxyphenyyl-rapamycin, 7-epi-thiomethyl-rapamycin, 7-demethoxy-rapamycin, 32-demethoxy-rapamycin, 7-desmethyl-rapamycin, 42-O-(2-hydroxy) ethyl-rapamycin, and other analogs of rapamycin.

12. A method of treating a cancer, a bacterial infection, a fungal infection, or a parasitic infection in a subject in need of such treatment, comprising administering to the subject a protein-drug conjugate comprising an annexin protein to which is covalently linked at least one therapeutic drug, wherein the therapeutic drug is an anticancer agent, an antibacterial antibiotic, an antifungal antibiotic, or an antiparasitic antibiotic, respectively.

13. The method of claim 12, wherein the annexin protein is human annexin A5.

14. The method of claim 12, further comprising administering a therapeutically-effective amount of at least one of an immunostimulant and an mTOR inhibitor to the subject.

15. The method of claim 14, wherein the mTOR inhibitor is selected from the group consisting of rapamycin, everolimus, temsirolimus, ridaforolimus, metformin, tacrolimus, ABT-578, AP23675, AP-23841, 7-epi-rapamycin, 7-thiomethyl-rapamycin, 7-epi-tromethoxyphenyyl-rapamycin, 7-epi-thiomethyl-rapamycin, 7-demethoxy-rapamycin, 32-demethoxy-rapamycin, 7-desmethyl-rapamycin, 42-O-(2-hydroxy) ethyl-rapamycin, and other analogs of rapamycin.

16-19. (canceled)

20. The method of claim 12, wherein the bacterial infection is an intracellular bacterial infection.

21-22. (canceled)

23. The method of claim 12, wherein the fungal infection is an intracellular fungal infection.

24-27. (canceled)

28. The method of claim 12, wherein the parasitic infection is an intracellular parasitic infection.

29-30. (canceled)

31. The protein-drug conjugate of claim 1, wherein the at least one therapeutic drug is an antifungal drug, and optionally, wherein the antifungal drug is selected from the group consisting of polyene antifungals, flucytosine, imidazole antifungals, triazole antifungals, and pharmaceutically acceptable salts thereof, and combinations thereof.

32. The method of claim 12, wherein the antifungal antibiotic is selected from the group consisting of polyene antifungals, flucytosine, imidazole antifungals, triazole antifungals, and pharmaceutically acceptable salts thereof, and combinations thereof.
